(19) United States
(12) Patent Application Publication
(10) Pub. No.: US 2020/0049682 A1
(43) Pub. Date: Feb. 13, 2020

(54) BALLAST WATER MEASUREMENT APPARATUS, SHIP COMPRISING BALLAST WATER MEASUREMENT APPARATUS, AND BALLAST WATER MEASUREMENT METHOD

(71) Applicant: KURITA WATER INDUSTRIES LTD., TOKYO (JP)

(72) Inventors: Kotaro FUKUZAWA, Tokyo (JP); Hiroshi MIYATA, Tokyo (JP)

(73) Assignee: KURITA WATER INDUSTRIES LTD., TOKYO (JP)

(21) Appl. No.: 16/492,615

(22) PCT Filed: Sep. 15, 2017
(86) PCT No.: PCT/JP2017/033517

§ 371 Date: Sep. 10, 2019

(30) Foreign Application Priority Data
Mar. 10, 2017 (JP) 2017-045747

(51) Int. Cl. G01N 33/18; B63B 13/00; B63J 4/00; C02F 1/66; C02F 1/70; C02F 1/72

(52) U.S. Cl. CPC G01N 33/18; B63B 13/00; B63J 4/002; C02F 2103/008; C02F 1/70; C02F 1/72; C02F 1/66

(57) ABSTRACT

An object of the present invention is to increase the accuracy of water quality measurement of ballast waters with different oxidant concentrations. A ballast water measurement apparatus (2 or 62) is provided with: a first measuring unit (6-1 or 64-1) for measuring an oxidant concentration of ballast water after addition of an oxidant or ballast water before addition of a neutralizing agent; a second measuring unit (6-2 or 64-2) for measuring an oxidant concentration of ballast water after neutralization of the oxidant; and a casing (4) that accommodates the first measuring unit and the second measuring unit. Oxidant concentration measurement ranges of the first measuring unit and the second measuring unit are different.

### TECHNICAL FIELD

The present invention relates to water quality measurement of ballast water loaded on a ship.

### BACKGROUND ART

In a ship such as a cargo ship, the amount of ballast water loaded on the ship is adjusted to control draft variation of the ship due to variation in the amount of cargo. This ballast water is loaded at a port of call where cargo is unloaded and discharged at a port of call where cargo is loaded. Ballast water supply treatment is known in which, in order to prevent marine pollution due to movement of aquatic life and pathogens included in ballast water, an oxidant, such as sodium hypochlorite and ozone (for example, Patent Document 1), calcium hypochlorite or sodium dichloroisocyanurate, is injected into the ballast water to sterilize the aquatic life and the pathogens. Further, in ballast water drainage treatment for discharging ballast water, treatment to neutralize ballast water by injecting a neutralizing agent is performed.

### PRIOR ART DOCUMENT

[Patent Document]

[Patent Document 1] JP2012-007969A

### SUMMARY OF THE INVENTION

### Problems to be Solved by the Invention

In the ballast water drainage treatment, for example, the water quality of ballast water after addition of an oxidant, into which the oxidant has been added at the time of the ballast water supply treatment, that is, ballast water before addition of a neutralizing agent (before oxidant neutralization) and the water quality of ballast water after oxidant neutralization are measured. Water quality information about the ballast water before neutralization is used, for example, to decide the amount of neutralizing agent to be injected, and water quality information about the ballast water after neutralization is used, for example, for water quality management of ballast water to be drained. In ballast water treatment accompanied by such water quality measurement, there is a problem in that, when water quality measurement of the ballast water before neutralization the oxidant concentration of which is high is considered, the accuracy of water quality measurement of the ballast water after neutralization the oxidant concentration of which is low or which does not include the oxidant decreases.

Therefore, an object of the present invention is to improve the accuracy of water quality measurement of ballast waters with different oxidant concentrations.

### Means for Solving the Problems

In order to achieve the above object, according to an aspect of a ballast water measurement apparatus of the present invention, there are provided a first measuring unit for measuring an oxidant concentration of ballast water after addition of an oxidant or ballast water before addition of a neutralizing agent; a second measuring unit for measuring an oxidant concentration of ballast water after neutralization of the oxidant; and a casing that accommodates the first measuring unit and the second measuring unit; and oxidant concentration measurement ranges of the first measuring unit and the second measuring unit only have to be different.

In the above ballast water measurement apparatus, there may be provided a switching unit that is connected to the first measuring unit and the second measuring unit and that is for switching supply destinations of ballast water supplied to a first supply pipe of the first measuring unit or a second supply pipe of the second measuring unit.

In the above ballast water measurement apparatus, both or one of the first measuring unit and the second measuring unit may include a flow rate adjusting unit for adjusting a flow rate of ballast water to be collected. The first measuring unit, the second measuring unit, or the first and second measuring units may measure an oxidant concentration of the ballast water adjusted by this flow rate adjusting unit.

In the above ballast water measurement apparatus, there may be provided a dilution water supplying unit that is connected to the first measuring unit and that is for supplying dilution water to the first measuring unit.

In the above ballast water measurement apparatus, the dilution water supplying unit may be provided with a dilution water pipe and a third flow rate adjusting unit for adjusting a flow rate of the dilution water flowing in the dilution water pipe.

In the above ballast water measurement apparatus, both or one of the first measuring unit and the second measuring unit may include a pressure adjusting unit. This pressure adjusting unit may adjust the pressure of ballast water in a water supply pipe of the first measuring unit or the second measuring unit.

In the above ballast water measurement apparatus, there may be provided a control unit for receiving both or one of a measurement result of the first measuring unit and a measurement result of the second measuring unit to generate an output signal of the both or one of the measurement results; and the output signal may include information about the oxidant concentration, adjustment information about the amount of the oxidant added, adjustment information about the amount of the neutralizing agent added for neutralizing the oxidant, alarm information or a signal to stop ballast water treatment.

In order to achieve the above object, according to an aspect of a ship provided with the ballast water measurement apparatus of the present invention, the ship only has to be provided with the ballast water measurement apparatus described above.

In order to achieve the above object, according to an aspect of a ballast water measurement method of the present invention, a process for measuring an oxidant concentration of ballast water after addition of an oxidant or ballast water before addition of a neutralizing agent, by a first measuring unit in a casing, and a process for measuring an oxidant concentration of ballast water after neutralization of the oxidant, by a second measuring unit in the casing are included; and oxidant concentration measurement ranges of the first measuring unit and the second measuring unit only have to be different.

### Effect of the Invention

According to the present invention, any of the following advantageous effects can be obtained.

(1) Since oxidant concentration measurement ranges of a first measuring unit and a second measuring unit are different, it is possible to measure each of ballast waters with different oxidant concentrations with a measuring unit suitable for the oxidant concentration of the ballast water and improve the accuracy of water quality measurement of the ballast water.

(2) It is possible to obtain a highly reliable water quality measurement result. For example, it is possible to grasp the oxidant concentration of ballast water discharged outside a ship under high reliability. When the oxidant concentration of ballast water discharged outside the ship is high, discharge of the oxidant into the sea can be controlled by treatment for reducing the oxidant concentration, and it is possible to control marine pollution by the oxidant.

Other objects, characteristics and advantages of the present invention will be clearer by referring to the accompanying drawings and embodiments.

### BRIEF DESCRIPTION OF DRAWINGS

FIG. 1 is a diagram illustrating an example of a ballast water measurement apparatus according to a first embodiment.

FIG. 2 is a diagram illustrating an example of a hardware configuration of a control unit.

FIG. 3 is a diagram illustrating an example of display of a water quality measurement result.

FIG. 4 is a flowchart illustrating an example of a water quality measurement process procedure.

FIG. 5 is a diagram illustrating an example of a switching unit according to a modification.

FIG. 6 is a diagram illustrating another example of the switching unit according to the modification.

FIG. 7 is a diagram illustrating an example of the ballast water measurement apparatus provided with a dilution water supplying unit.

FIG. 8 is a diagram illustrating another example of the ballast water measurement apparatus provided with the dilution water supplying unit.

FIG. 9 is a diagram illustrating an example of the ballast water measurement apparatus provided with a pressure adjusting unit and a backflow preventing unit.

FIG. 10 is a diagram illustrating an example of a ballast water measurement apparatus according to a second embodiment.

FIG. 11 is a diagram illustrating a measuring instrument and pipes connected to the measuring instrument.

FIG. 12 is a flowchart illustrating an example of a water quality measurement process procedure.

FIG. 13 is a flowchart illustrating an example of a process procedure for cleaning treatment of a measuring unit.

FIG. 14 is a diagram illustrating an example of connection between the ballast water measurement apparatus and ballast water treatment equipment.

FIG. 15 is a diagram illustrating an example of a treatment sequence of the ballast water measurement apparatus and the ballast water treatment equipment.

FIG. 16 is a diagram illustrating another example of the treatment sequence of the ballast water measurement apparatus and the ballast water treatment equipment.

### EMBODIMENTS FOR CARRYING OUT THE INVENTION

### First Embodiment

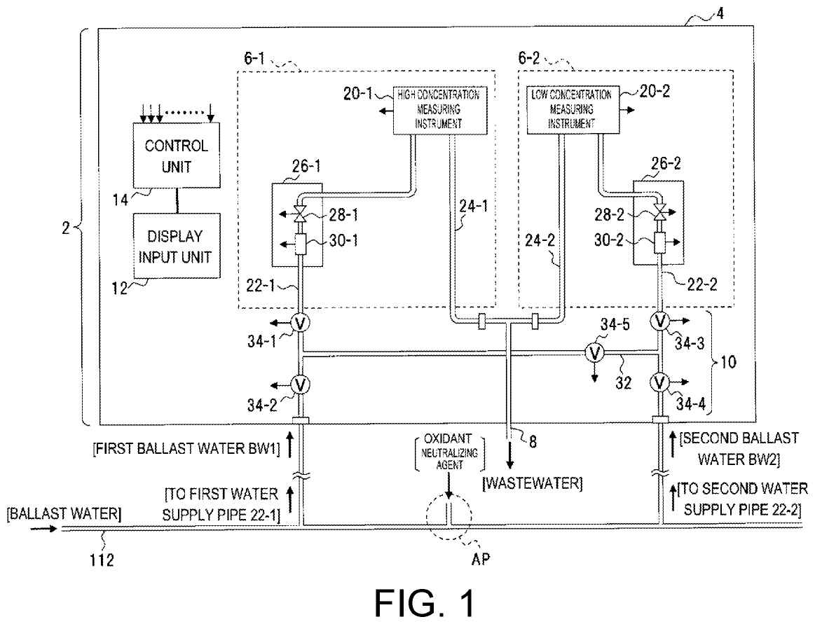

FIG. 1 will be referred to about a first embodiment. FIG. 1 illustrates an example of a ballast water measurement apparatus according to the first embodiment. In FIG. 1, thin arrows attached to a high concentration measuring instrument, a low concentration measuring instrument, a flow rate adjusting unit, a switching unit and a control unit indicate connections between the control unit and the devices, and thick arrows attached to an inlet of a water supply pipe, an outlet of a draining unit and a water treatment line indicate flows of ballast water, wastewater, an oxidant or a neutralizing agent.

A ballast water measurement apparatus 2 (hereinafter referred to as “the measurement apparatus 2”) is an example of a measurement apparatus for measuring water qualities of a plurality of ballast waters, for example, concentrations of an oxidant included in the ballast waters, such as sodium hypochlorite, ozone, calcium hypochlorite or sodium dichloroisocyanurate. The measurement apparatus 2 is provided with a casing 4, a first measuring unit 6-1 (hereinafter referred to as “the measuring unit 6-1”), a second measuring unit 6-2 (hereinafter referred to as “the measuring unit 6-2”), a draining unit 8, a switching unit 10, a display input unit 12 and a control unit 14. First ballast water BW1 (hereinafter referred to as “the ballast water BW1”) and second ballast water BW2 (hereinafter referred to as “the ballast water BW2”) are supplied to the measurement apparatus 2. The ballast waters BW1 and BW2 are collected from ballast water flowing through a water treatment line 112 of ballast water treatment equipment installed in a ship. The water treatment line 112 is provided with a chemical addition position AP at which the oxidant or the neutralizing agent is added. The ballast water BW1 is ballast water upstream of the chemical addition position AP and is ballast water before addition of the oxidant or before addition of the neutralizing agent, that is, ballast water immediately before being discharged outside the ship, to which the oxidant has been added. The ballast water BW2 is ballast water downstream of the chemical addition position AP and is ballast water after addition of the oxidant or ballast water after addition of the neutralizing agent (that is, after oxidant neutralization).

The casing 4 accommodates the measuring unit 6-1, the measuring unit 6-2, the draining unit 8, the switching unit 10, the display input unit 12 and the control unit 14, and these units are put together in the casing. This casing 4 is, for example, a metal casing and gives rigidity to the measurement apparatus 2.

The measuring unit 6-1 is provided with a high concentration measuring instrument 20-1, a first water supply pipe 22-1 (hereinafter referred to as “the water supply pipe 22-1”), a first drainage pipe 24-1 (hereinafter referred to as “the drainage pipe 24-1”) and a first flow rate adjusting unit 26-1 (hereinafter referred to as “the flow rate adjusting unit 26-1”). The measuring unit 6-1 measures the oxidant concentration of ballast water supplied from the water supply pipe 22-1 by the high concentration measuring instrument 20-1. The measuring unit 6-1 has a high concentration measurement range so as to be suitable for water quality measurement of ballast water after addition of the oxidant. In the present specification, it is assumed that the term “high concentration” indicates being higher than the term “low concentration” described later. The term “low concentration” means, for example, the oxidant concentration of ballast water after oxidant neutralization and the oxidant concentration of ballast water before addition of the oxidant, that is, a concentration 0 or a concentration at a level similar to that of sea water. The term “high concentration” means, for example, a concentration higher than the already described low concentration up to the concentration of ballast water after addition of the oxidant. This term “high concentration” may include a concentration higher than the concentration of the ballast water after addition of the oxidant. Further, in the present specification, it is assumed that the term “high concentration measurement range” indicates that the measurement range includes a range higher than a term “low concentration measurement range” described later. The term “low concentration measurement range” only has to include the already described low concentration, and the term “high concentration measurement range” only has to include the already described high concentration. The low and high concentration measurement ranges only have to at least partially overlap with each other. Though the measuring units 6-1 and 6-2 are capable of measuring particular concentrations, their concentration measurement ranges are treated as being different from each other if the measurement accuracy of one of the measuring units 6-1 and 6-2 has not reached a practical level.

The high concentration measuring instrument 20-1 is connected to the water supply pipe 22-1 and the drainage pipe 24-1. The high concentration measuring instrument 20-1, the water supply pipe 22-1 and the drainage pipe 24-1 form a first ballast water flow path. The flow rate adjusting unit 26-1 is installed in the water supply pipe 22-1. This flow rate adjusting unit 26-1 is provided with an on-off valve 28-1 (for example, a solenoid valve) and a constant flow rate valve 30-1. The flow rate adjusting unit 26-1 supplies a certain amount of ballast water or stops supply of the ballast water. That is, the flow rate adjusting unit 26-1 adjusts the flow rate of ballast water flowing through the water supply pipe 22-1.

The high concentration measuring instrument 20-1 is an example of a first measuring instrument for measuring the water quality of ballast water supplied from the water supply pipe 22-1. The high concentration measuring instrument 20-1 measures the oxidant concentration of ballast water, for example, a TRO (Total Residual Oxidants) concentration. The concentration measurement range of the high concentration measuring instrument 20-1 only has to include the oxidant concentration of ballast water to which the oxidant is added, and concentrations near the concentration and is, for example, 0.5 to 4.0 [mg/L]. It is possible to measure a concentration exceeding 4.0 [mg/L] by appropriate dilution. The high concentration measuring instrument 20-1 only has to be capable of measuring the oxidant concentration of ballast water. For example, the high concentration measuring instrument 20-1 includes a colorimeter so that light absorption strength of ballast water colored into pink to pinkish red according to a TRO concentration by addition of a DPD (diethyl-p-phenylenediamine) reagent can be measured by a DPD colorimetric method or a DPD absorption spectrophotometry.

The water supply pipe 22-1 supplies ballast water to the high concentration measuring instrument 20-1, and the drainage pipe 24-1 drains the ballast water measured by the high concentration measuring instrument 20-1 to the draining unit 8. The water supply pipe 22-1 and the drainage pipe 24-1 only have to be pipes having corrosion resistance against the oxidant included in ballast water and only have to be, for example, resin pipes such as fluororesin pipes or vinyl chloride pipes, stainless pipes or corrosion-prevention-treated metal pipes.

The measuring unit 6-2 is provided with a low concentration measuring instrument 20-2, a second water supply pipe 22-2 (hereinafter referred to as “the water supply pipe 22-2”), a second drainage pipe 24-2 (hereinafter referred to as “the drainage pipe 24-2”) and a second flow rate adjusting unit 26-2 (hereinafter referred to as “the flow rate adjusting unit 26-2”). This measuring unit 6-2 measures the oxidant concentration of ballast water supplied from the water supply pipe 22-2 by the high concentration measuring instrument 20-2. The measuring unit 6-2 has a low concentration measurement range so as to be suitable for water quality measurement of ballast water after oxidant neutralization and ballast water before addition of the oxidant. The low concentration measuring instrument 20-2 is connected to the water supply pipe 22-2 and the drainage pipe 24-2. The low concentration measuring instrument 20-2, the water supply pipe 22-2 and the drainage pipe 24-2 form a second ballast water flow path. The flow rate adjusting unit 26-2 is installed in the water supply pipe 22-2. This flow rate adjusting unit 26-2 is provided with an on-off valve 28-2 (for example, a solenoid valve) and a constant flow rate valve 30-2. The flow rate adjusting unit 26-2 supplies a certain amount of ballast water or stops supply of the ballast water. That is, the flow rate adjusting unit 26-2 adjusts the flow rate of ballast water flowing through the water supply pipe 22-2.

The low concentration measuring instrument 20-2 is an example of a second measuring instrument for measuring the water quality of ballast water supplied from the water supply pipe 22-2. The low concentration measuring instrument 20-2 measures the oxidant concentration of ballast water, for example, a TRO concentration. The concentration measurement range of the low concentration measuring instrument 20-2 only has to include the oxidant concentrations of ballast water after oxidant neutralization and ballast water before addition of the oxidant, and concentrations near the concentrations and is, for example, 0.0 to 2.0 [mg/L]. The low concentration measuring instrument 20-2 includes, for example, the already described colorimeter so that light absorption strength of ballast water colored according to a TRO concentration by addition of a DPD reagent can be measured by a DPD colorimetric method or a DPD absorption spectrophotometry.

The water supply pipe 22-2 supplies ballast water to the low concentration measuring instrument 20-2, and the drainage pipe 24-2 drains the ballast water measured by the low concentration measuring instrument 20-2 to the draining unit 8. The water supply pipe 22-2 and the drainage pipe 24-2 only have to be pipes having corrosion resistance against the oxidant included in ballast water and only have to be, for example, resin pipes such as fluororesin pipes or vinyl chloride pipes, stainless pipes or corrosion-prevention-treated metal pipes.

The draining unit 8 is an example of means for conveying wastewater. The draining unit 8 includes a branch pipe extending to the outside of the casing 4 and in two directions inside the casing and forms outlet piping for ballast water discharged from the measuring units 6-1 and 6-2. One of the pipes extending in the two directions in the casing is connected to the drainage pipe 24-1 of the measuring unit 6-1, and the other one of the pipes extending in the two directions in the casing is connected to the drainage pipe 24-2 of the measuring unit 6-2. The draining unit 8 causes ballast waters discharged from the measuring units 6-1 and 6-2 to join and collectively discharges the ballast waters to the outside of the casing 4. The draining unit 8 only has to be a pipe having corrosion resistance against the oxidant included in ballast water and only has to be, for example, a resin pipe such as a fluororesin pipe or a vinyl chloride pipe, a stainless pipe or a corrosion-prevention-treated metal pipe.

The measuring units 6-1 and 6-2 are arranged, for example, symmetrically relative to an extension passing through the draining unit 8. By symmetrically arranging the measuring units 6-1 and 6-2, it is not necessary to separately grasp placements of the measuring units 6-1 and 6-2, so that a burden of handling the measurement apparatus 2 is reduced.

The switching unit 10 is provided with a connection pipe 32 and switching valves 34-1, 34-2, 34-3, 34-4 and 34-5. The connection pipe 32 is connected to the water supply pipe 22-1 of the measuring unit 6-1 and the water supply pipe 22-2 of the measuring unit 6-2 to cause the water supply pipe 22-1 and the water supply pipe 22-2 to communicate with each other. The switching valve 34-1 is installed in the water supply pipe 22-1 on the downstream side of a first connecting section of the connection pipe 32 and the water supply pipe 22-1, and the switching valve 34-2 is installed in the water supply pipe 22-1 on the upstream side of the first connecting section. The switching valve 34-3 is installed in the water supply pipe 22-2 on the downstream side of a second connecting section of the connection pipe 32 and the water supply pipe 22-2, and the switching valve 34-4 is installed in the water supply pipe 22-2 on the upstream side of the second connecting section. The switching valve 34-5 is installed in the connection pipe 32. The switching valves 34-1, 34-2, 34-3, 34-4 and 34-5 cause ballast water to pass through or shut off the ballast water by opening or closing. The switching unit 10 switches supply destinations of the ballast water BW1 supplied to the water supply pipe 22-1 and the ballast water BW2 supplied to the water supply pipe 22-2 to the high concentration measuring instrument 20-1 of the measuring unit 6-1 or the low concentration measuring instrument 20-2 of the measuring unit 6-2. For example, when the switching valves 34-1, 34-4 and 34-5 are opened, and the switching valves 34-2 and 34-3 are closed, the switching unit 10 supplies the ballast water BW2 supplied to the water supply pipe 22-2 to the measuring unit 6-1. When the switching valves 34-1, 34-2, 34-3 and 34-4 are opened, and the switching valves 34-5 is closed, the switching unit 10 supplies the ballast water BW1 supplied to 22-1 to the measuring unit 6-1 and supplies the ballast water BW2 supplied to the water supply pipe 22-2 to the measuring unit 6-2. By switching by the switching unit 10, a supply destination of ballast water can be changed according to the oxidant concentration of the ballast water.

The display input unit 12 displays information based on an output of the control unit 14 and generates instruction information to the measurement apparatus 2 in response to an operation. The display input unit 12 includes a display, for example, a liquid crystal display, an LED (light emitting diode) display or an organic electroluminescent (EL) display. The display input unit 12 displays, for example, water quality information about the ballast waters BW1 and BW2, and measurement apparatus information such as operation information about the measurement apparatus 2 and alarm information in response to an output of the control unit 14. Furthermore, the display input unit 12 includes, for example, a touch panel and generates setting information about the measurement apparatus 2 or instruction information such as display switching information in response to an operation.

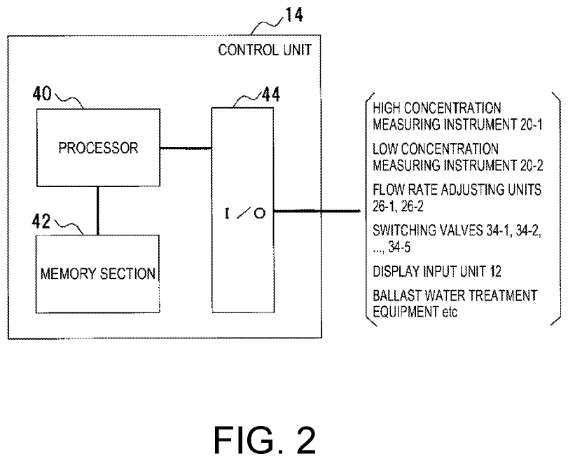

The control unit 14 is an example of a computer provided with a water quality measurement function, a measurement result output function and a function of communicating with external apparatuses. FIG. 2 illustrates an example of a hardware configuration of the control unit 14. The control unit 14 is provided with a processor 40, a memory section 42 and an I/O (input-output) 44.

The processor 40 is an example of an information processing unit for processing information and is, for example, a CPU (central processing unit). The processor 40 executes an OS (operating system) and a water quality measurement program stored in the memory section 42 to perform various kinds of information processing. The information processing executed by the processor 40 includes an instruction to adjust flow rates of the flow rate adjusting units 26-1 and 26-2, an instruction to open/close the switching valves 34-1, 34-2, 34-3, 34-4 and 34-5, an instruction to operate or stop the high concentration measuring instrument 20-1 and the low concentration measuring instrument 20-2 (hereinafter, the high concentration measuring instrument 20-1 and the low concentration measuring instrument 20-2 will be collectively referred to as “the measuring instruments 20-1 and 20-2), processing of measurement data obtained by the measuring instruments 20-1 and 20-2, output of measurement apparatus information, processing of instruction information generated by the display input unit 12, and input/output control at the I/O 44.

The memory section 42 stores therein programs such as the OS and the water quality measurement program to be executed by the processor 40. Storage or readout of various kinds of information to or from the memory section 42 is performed by control of the processor 40. The memory section 42 is provided with one or more storage elements such as a ROM (read-only memory), a RAM (random-access memory), an EEPROM (electrically erasable programmable read-only memory), a NAND-type flash memory and a NOR-type flash memory. A hard disk device or a semiconductor storage device may be used as the storage element.

The I/O 44 wiredly or wirelessly connects to connected equipment such as the measuring instruments 20-1 and 20-2, the flow rate adjusting units 26-1 and 26-2, the switching valves 34-1, 34-2, 34-3, 34-4 and 34-5, the display input unit 12 and the ballast water treatment equipment. The I/O 44 is used for transmission/reception of data between the processor 40 and the connected equipment.

[Display of Water Quality Measurement Result]

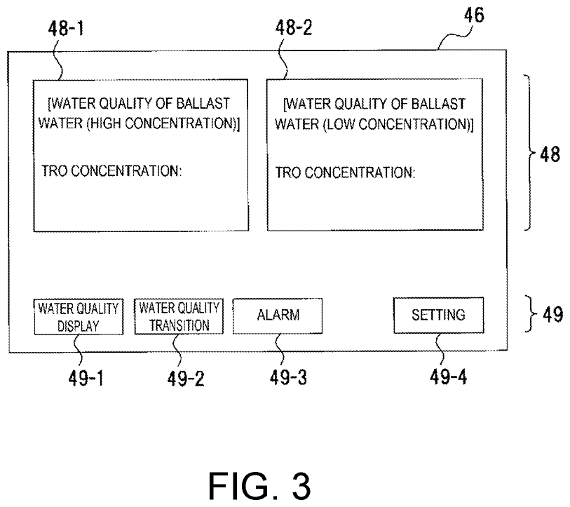

Next, FIG. 3 will be referred to about display of water quality measurement results of the ballast waters BW1 and BW2. FIG. 3 illustrates an example of display of water quality measurement results. The display input unit 12 displays this display screen 46 based on a display instruction outputted by the control unit 14.

This display screen 46 includes a display area 48 and an operation area 49. Information of a selection item selected by an operation of the operation area 49 is displayed in the display area 48. For example, when a selection item “water quality display” is selected, the display area 48 which includes, for example, a first display area 48-1 and a second display area 48-2 is displayed. Information about the water quality of ballast water measured by the measuring unit 6-1 is displayed in the first display area 48-1. Information about the water quality of ballast water measured by the measuring unit 6-2 is displayed in the second display area 48-2. In the first display area 48-1, the water quality of the ballast water, for example, a TRO concentration is displayed, for example, together with a display item “water quality of ballast water (high concentration)”. In the second display area 48-2, the water quality of the ballast water, for example, a TRO concentration is displayed, for example, together with a display item “water quality of ballast water (low concentration)”.

In the operation area 49, operation buttons for selection items to be displayed in the display area 48 are displayed. The operation buttons include, for example, a selection button 49-1 to cause the selection item “water quality display” to be displayed, a selection button 49-2 to cause a selection item “water quality transition” to be displayed, a selection button 49-3 to cause a selection item “alarm” to be displayed and a selection button 49-4 to cause a selection item “setting” to be displayed. An example in which the quality information is displayed in the display area 48 is illustrated in FIG. 3. However, transition of the measured water quality of ballast water can be displayed in the display area 48 when the selection item “water quality transition” is selected; alarm information, for example, an alarm being generated or an alarm history can be displayed in the display area 48 when the selection item “alarm” is selected; and set items of the measurement apparatus 2 can be displayed in the display area 48 when the selection item “setting” is selected.

[Process Procedure for Water Quality Measurement of Ballast Waters BW1 and BW2]

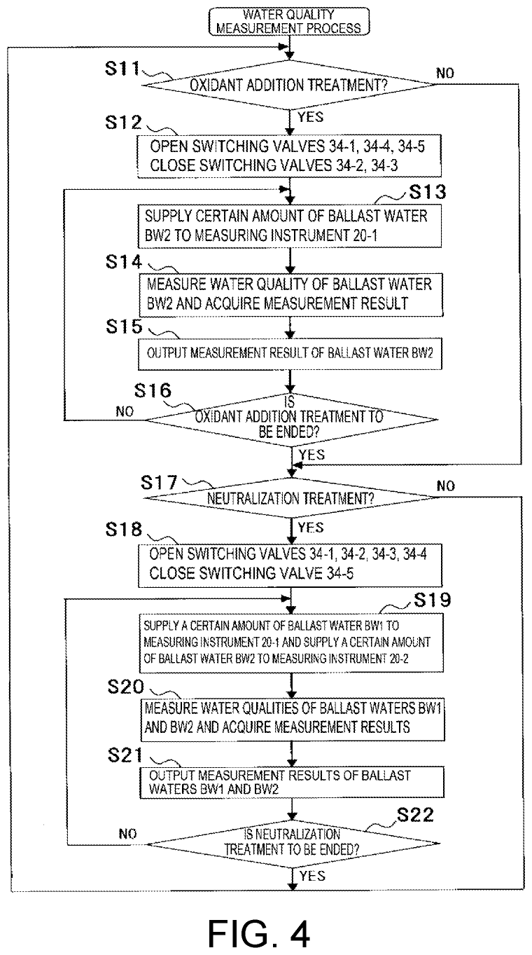

Next, FIG. 4 will be referred to about a process procedure for water quality measurement of the ballast waters BW1 and BW2. FIG. 4 is a flowchart illustrating an example of a water quality measurement process procedure. This water quality measurement process procedure is an example of a ballast water measurement method of the present invention and is performed by the control unit 14. In FIG. 4, step S indicates a process stage.

The control unit 14 judges whether oxidant addition treatment is to be performed (step S11). At the time of ballast water supply treatment for loading ballast water into the ship, the control unit 14 can, for example, acquire ballast water treatment information indicating the ballast water supply treatment from the ballast water treatment equipment installed in the ship and judge that the oxidant addition treatment is to be performed. At the time of ballast water drainage treatment for discharging ballast water from the ship, the control unit 14 can, for example, acquire ballast water treatment information indicating the ballast water drainage treatment from the ballast water treatment equipment and judge that neutralization treatment is to be performed. If the oxidant addition treatment is to be performed (step S11: YES), the control unit 14 opens the switching valves 34-1, 34-4 and 34-5 and closes the switching valves 34-2 and 34-3 (step S12). After that, the control unit 14 performs a first water quality measurement process (steps S13 to S15). The first water quality measurement process is an example of a water quality measurement process in the oxidant addition treatment. In this first water quality measurement process, the control unit 14 causes the flow rate adjusting unit 26-1 to operate to supply a certain amount of ballast water BW2, that is, ballast water after addition of the oxidant to the high concentration measuring instrument 20-1 (step S13). The control unit 14 causes the high concentration measuring instrument 20-1 to measure the water quality of the ballast water BW2 and acquires a result of the measurement of the ballast water BW2 from the high concentration measuring instrument 20-1 (step S14). The ballast water BW2 after measurement is discharged from the draining unit 8. Further, the control unit 14 outputs the acquired measurement result, for example, to the display input unit 12 and the ballast water treatment equipment (step S15). The control unit 14 judges whether the oxidant addition treatment is to be ended (step S16). If the oxidant addition treatment is not to be ended (step S16: NO), the control unit 14 returns to step S13 and repeats the first water quality measurement process (steps S13 to S15) and step S16. The control unit 14 can judge end of the oxidant addition treatment by cease of the ballast water treatment information indicating the ballast water supply treatment or by acquisition of ballast water treatment information indicating end of the ballast water supply treatment from the ballast water treatment equipment. If the oxidant addition treatment is not to be performed (step S11: NO), steps S12 to S16 are omitted.

If the oxidant addition treatment is not to be performed (step S11: NO) or if the oxidant addition treatment is to be ended (step S16: YES), the control unit 14 judges whether the neutralization treatment is to be performed (step S17). If the neutralization treatment is to be performed (step S17: YES), the control unit 14 opens the switching valves 34-1, 34-2, 34-3 and 34-4 and closes the switching valve 34-5 (step S18). After that, the control unit 14 performs a second water quality measurement process (steps S19 to S21). The second water quality measurement process is an example of a water quality measurement process in the neutralization treatment. In this second water quality measurement process, the control unit 14 causes the flow rate adjusting units 26-1 and 26-2 to operate to supply a certain amount of ballast water BW1, that is, ballast water after addition of the oxidant (before addition of the neutralizing agent) to the high concentration measuring instrument 20-1 and supply a certain amount of ballast water BW2, that is, ballast water after oxidant neutralization to the low concentration measuring instrument 20-2 (step S19). The control unit 14 causes the high concentration measuring instrument 20-1 to measure the water quality of the ballast water BW1 and acquire a result of the measurement of the ballast water BW1 from the high concentration measuring instrument 20-1, and causes the low concentration measuring instrument 20-2 to measure the water quality of the ballast water BW2 and acquire a result of the measurement of the ballast water BW2 from the low concentration measuring instrument 20-2 (step S20). The ballast waters BW1 and BW2 after measurement are discharged from the draining unit 8. Further, the control unit 14 outputs the acquired measurement result, for example, to the display input unit 12 and the ballast water treatment equipment (step S21). The control unit 14 judges whether the neutralization treatment is to be ended (step S22). If the neutralization treatment is not to be ended (step S22: NO), the control unit 14 returns to step S19 and repeats the second water quality measurement process (steps S19 to S21) and step S22. The control unit 14 can judge end of the oxidant neutralization treatment by cease of ballast water treatment information indicating the ballast water drainage treatment or by acquisition of ballast water treatment information indicating end of the ballast water drainage treatment from the ballast water treatment equipment. If the neutralization treatment is not to be performed (step S17: NO), steps S18 to S22 are omitted.

If the neutralization treatment is not to be performed (step S17: NO), or if the neutralization treatment is to be ended (step S22: YES), the control unit 14 returns to step S11, repeats this process procedure and can continuously or intermittently measure the water qualities of the ballast waters BW1 and BW2. Since the process is performed individually for each of the measuring units 6-1 and 6-2, the control unit 14 can not only perform water quality measurement before and after the ballast water supply treatment or the ballast water drainage process but also perform water quality measurement either before or after the treatment.

[Advantageous Effects of First Embodiment]

(1) It is possible to measure ballast waters with different oxidant concentrations by measuring units suitable for measurement of the ballast waters, respectively, and it is possible to increase the accuracy of water quality measurement of the ballast waters.

(2) It is possible to obtain a highly reliable water quality measurement result. For example, it is possible to grasp the oxidant concentration of ballast water discharged outside the ship under high reliability. When the oxidant concentration of the ballast water discharged outside the ship is high, the amount of oxidant discharged can be controlled by treatment for reducing the oxidant concentration, and it is possible to control marine pollution by the oxidant.

(3) Since the measurement apparatus 2 is provided with the switching unit 10, ballast water can be supplied to each of the water supply pipes 22-1 and 22-2 irrespective of the water quality of the ballast water. Therefore, since ballast water after addition of the oxidant or before addition of the neutralizing agent is supplied to the high concentration measuring instrument 20-1, and ballast water after oxidant neutralization or ballast water before addition of the oxidant is supplied to the low concentration measuring instrument 20-2, it is not necessary to cause directions of ballast water in the water treatment line 112 to be different in the ballast water supply treatment and in the ballast water drainage treatment. It is possible to increase a degree of freedom of connection between the water treatment line 112 of the ballast water treatment equipment and the measurement apparatus 2.

(4) Since the measurement apparatus 2 is provided with the flow rate adjusting units 26-1 and 26-2, it is possible to supply a certain amount of ballast water to the measuring instruments 20-1 and 20-2, and it is possible to increase stability of water quality measurement by the measuring instruments 20-1 and 20-2.

(5) It is possible to separately measure water qualities of ballast waters at two positions by one measurement apparatus 2. At the time of the ballast water drainage treatment, for example, water qualities of ballast waters before and after injection of the neutralizing agent can be measured by one measurement apparatus. That is, it is possible, in the ship, to control the number of water quality measurement apparatuses for ballast water installed together with the ballast water treatment equipment.

If the ballast water treatment equipment acquires oxidant concentration information about ballast water before injection of the neutralizing agent, the ballast water treatment equipment can adjust the amount of neutralizing agent to be injected, according to this concentration information. If the ballast water treatment equipment acquires oxidant concentration information about ballast water after injection of the neutralizing agent, the ballast water treatment equipment can grasp or record the oxidant concentration of ballast water before drainage.

(6) Since the draining unit 8, the display input unit 12 and the control unit 14 are shared by the two measuring units 6-1 and 6-2, an area required for installation of these members is reduced. Therefore, it is possible to reduce an area for installing the measurement apparatus 2 and reduce a work area required for operation or maintenance of the measurement apparatus 2.

(7) Since water qualities of ballast waters at two positions are measured by one measurement apparatus, a burden of cooperation between the ballast water treatment equipment and the measurement apparatus 2 is reduced. That is, in the case of measuring water qualities of ballast waters at two positions in ballast water intake treatment or drainage treatment, it is not necessary to cause the ballast water treatment equipment to cooperate with a plurality of measurement apparatuses, and cooperation is easy.

(8) Since the number of measurement apparatuses 2 is smaller than the number of positions of water quality measurement of ballast water, a burden of equipment management on the measurement apparatus 2 is reduced.

(9) Water qualities of ballast waters at two positions can be displayed on the display input unit 12 of one measurement apparatus 2 side by side. For example, a manager of the ballast water treatment equipment can compare water qualities before and after ballast water treatment by a chemical such as the oxidant or the neutralizing agent shown side by side and can efficiently grasp the water qualities of the ballast waters.

[Modification]

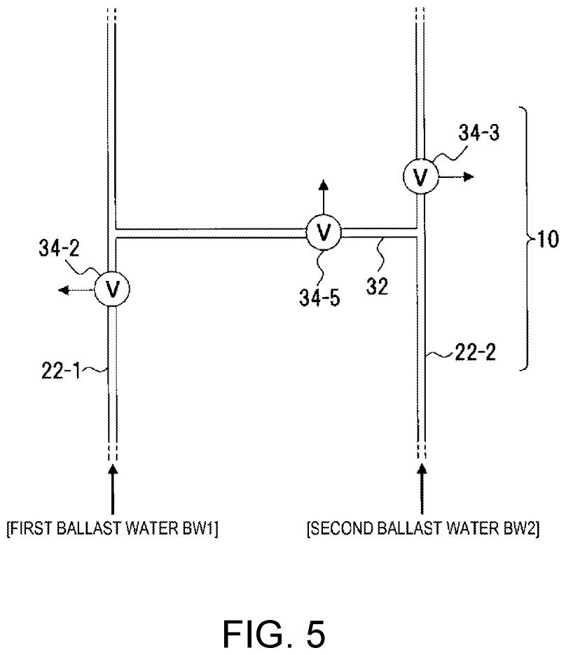

(1) In the above embodiment, the switching unit 10 is provided with the connection pipe 32 and the switching valves 34-1, 34-2, 34-3, 34-4 and 34-5 and switches the supply destinations of the ballast waters BW1 and BW2 to the high concentration measuring instrument 20-1 of the measuring unit 6-1 or the low concentration measuring instrument 20-2 of the measuring unit 6-2. However, the switching unit 10 only has to switch the supply destination of the ballast water BW2 to the high concentration measuring instrument 20-1 or the low concentration measuring instrument 20-2 and is not limited to the above embodiment. As illustrated in FIG. 5, the switching unit 10 may be provided with the already described connection pipe 32 and switching valves 34-2, 34-3 and 34-5 described above. In the case of the switching unit 10 illustrated in FIG. 5, the switching unit 10 can supply the ballast water BW2 to the measuring unit 6-1 when the switching valve 34-5 is opened, and the switching valves 34-2 and 34-3 are closed, and can supply the ballast water BW1 to the measuring unit 6-1 and supply the ballast water BW2 to the measuring unit 6-2 when the switching valves 34-2 and 34-3 are opened, and the switching valve 34-5 is closed. That is, ballast water including the oxidant before neutralization can be supplied to the high concentration measuring instrument 20-1, and ballast water after neutralization can be supplied to the low concentration measuring instrument 20-2. In the switching unit 10 illustrated in FIG. 5, the connection pipe 32 may be directly connected to the flow rate adjusting unit 26-1 or the high concentration measuring instrument 20-1 instead of the water supply pipe 22-1.

A supply destination of ballast water can also be switched by providing three-way valves in the first and second connecting sections between the connection pipe 32 and the water supply pipes 22-1 and 22-2 instead of the switching valves 34-1, 34-2, 34-3, 34-4 and 34-5.

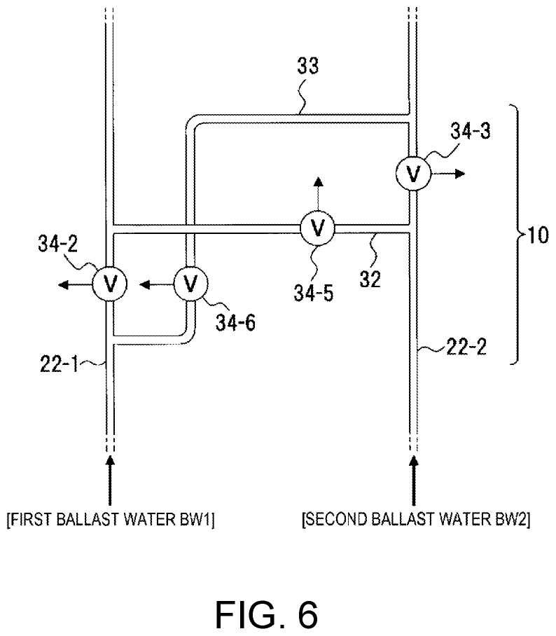

(2) As illustrated in FIG. 6, the switching unit 10 may be provided with a connection pipe 33 and a switching valve 34-6 in addition to the already described connection pipe 32 and switching valves 34-2, 34-3 and 34-5. The connection pipe 33 is connected to the water supply pipe 22-1 on the upstream side of an installation position of the switching valve 34-2 and to the water supply pipe 22-2 on the downstream side of an installation position of the switching valve 34-3. The switching valve 34-6 is installed in the connection pipe 33. That is, the switching valve 34-2 is located between a connecting section between the connection pipe 32 and the water supply pipe 22-1 and a connecting section between the connection pipe 33 and the water supply pipe 22-1, and the switching valve 34-3 is located between a connecting section between the connection pipe 32 and the water supply pipe 22-2 and a connecting section between the connection pipe 33 and the water supply pipe 22-2. In the case of the switching unit 10 illustrated in FIG. 6, the switching unit 10 can supply the ballast water BW2 to the measuring unit 6-1 when the switching valve 34-5 is opened, and the switching valves 34-2, 34-3 and 34-6 are closed, and can supply the ballast water BW1 to the measuring unit 6-1 and supply the ballast water BW2 to the measuring unit 6-2 when the switching valves 34-2 and 34-3 are opened, and the switching valves 34-5 and 34-6 are closed. That is, ballast water including the oxidant before neutralization can be supplied to the high concentration measuring instrument 20-1, and ballast water after neutralization can be supplied to the low concentration measuring instrument 20-2. Furthermore, in the case of the switching unit 10 illustrated in FIG. 6, the switching unit 10 can supply the ballast water BW2 to the measuring unit 6-1 and supply the ballast water BW1 to the measuring unit 6-2 when the switching valves 34-5 and 34-6 are opened, and the switching valves 34-2 and 34-3 are closed. In the switching unit 10 illustrated in FIG. 6, a degree of freedom of switching is increased. In the switching unit 10 illustrated in FIG. 6, the connection pipe 32 may be directly connected to the flow rate adjusting unit 26-1 or the high concentration measuring instrument 20-1 instead of the water supply pipe 22-1, and the connection pipe 33 may be directly connected to the flow rate adjusting unit 26-2 or the low concentration measuring instrument 20-2 instead of the water supply pipe 22-2.

(3) Though the switching unit 10 is used to measure the water quality of ballast water after addition of the oxidant by the measuring unit 6-1 in the above embodiment, the switching unit 10 can be used for various purposes. For example, when it is assumed that, in the ballast water supply treatment, the switching unit 10 supplies ballast water before addition of the oxidant to the measuring units 6-1 and 6-2, the switching unit 10 may use this ballast water before addition of the oxidant to perform a calibration process, for example, a 0-point correction process for the high concentration measuring instrument 20-1 and the low concentration measuring instrument 20-2. The calibration process for the measuring instruments 20-1 and 20-2 can be performed, for example, before starting the oxidant addition treatment, and reliability of a measurement result can be secured by this calibration process. This calibration process may be performed by supplying the ballast water BW2 to the measuring instruments 20-1 and 20-2 before starting addition of the oxidant to the ballast water.

(4) In the above embodiment, the flow rate adjusting units 26-1 and 26-2 are provided with the on-off valves 28-1 and 28-2 and the constant flow rate valves 30-1 and 30-2. The flow rate adjusting units 26-1 and 26-2 only have to be capable of supplying a certain amount of ballast water to the measuring instruments 20-1 and 20-2. For example, the flow rate adjusting units 26-1 and 26-2 may include metering pumps, and a certain amount of ballast water may be supplied to the measuring instruments 20-1 and 20-2 by these metering pumps.

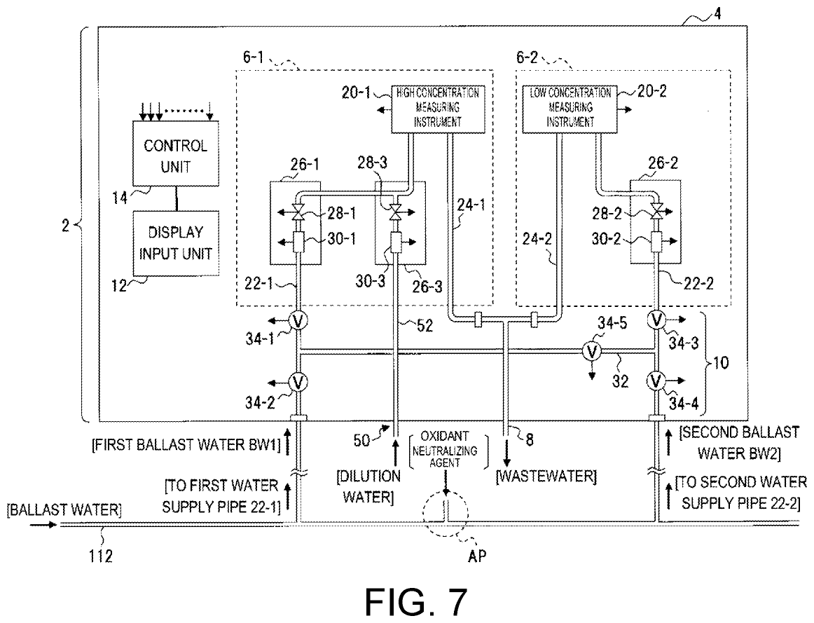

(5) As illustrated in FIG. 7, the measurement apparatus 2 may be provided with a dilution water supplying unit 50. The dilution water supplying unit 50 is provided with a dilution water pipe 52 and a third flow rate adjusting unit 26-3 (hereinafter referred to as “the flow rate adjusting unit 26-3”). The dilution water pipe 52 is an example of means for conveying dilution water. The dilution water pipe 52 extends from outside the casing 4 and is connected to the water supply pipe 22-1 on the downstream side of an installation position of the flow rate adjusting unit 26-3. The dilution water pipe 52 only has to be a pipe that does not cause material change due to contact with water and is, for example, a resin pipe such as a fluororesin pipe or a vinyl chloride pipe, a stainless pipe, a corrosion-prevention-treated metal pipe or the like. The flow rate adjusting unit 26-3 is installed in the dilution water pipe 52. The flow rate adjusting unit 26-3 is provided with an on-off valve 28-3 (for example, a solenoid valve) and a constant flow rate valve 30-3. The flow rate adjusting unit 26-3 supplies a certain amount of dilution water or stops supply of the dilution water. That is, the flow rate adjusting unit 26-3 adjusts the flow rate of dilution water to be supplied to the water supply pipe 22-1. The dilution water supplying unit 50 supplies dilution water with a certain flow rate to ballast water which has been flow-rate-adjusted by the flow rate adjusting unit 26-1 to dilute the ballast water at a certain rate. When the oxidant concentration temporarily increases in the ballast water supply treatment, it is possible to prevent water quality measurement outside the concentration measurement range of the high concentration measuring instrument 20-1 by measuring diluted ballast water. For example, if four-fold dilution water is supplied to ballast water the concentration of which is 20 [mg/L], the concentration of the ballast water can be measured by the high concentration measuring instrument 20-1 the concentration measurement range of which is 0.5 to 4.0 [mg/L]. In the case of measuring the water quality of diluted ballast water, the oxidant concentration of ballast water before the dilution can be determined by a calculation process by the control unit 14 in consideration of the dilution rate.

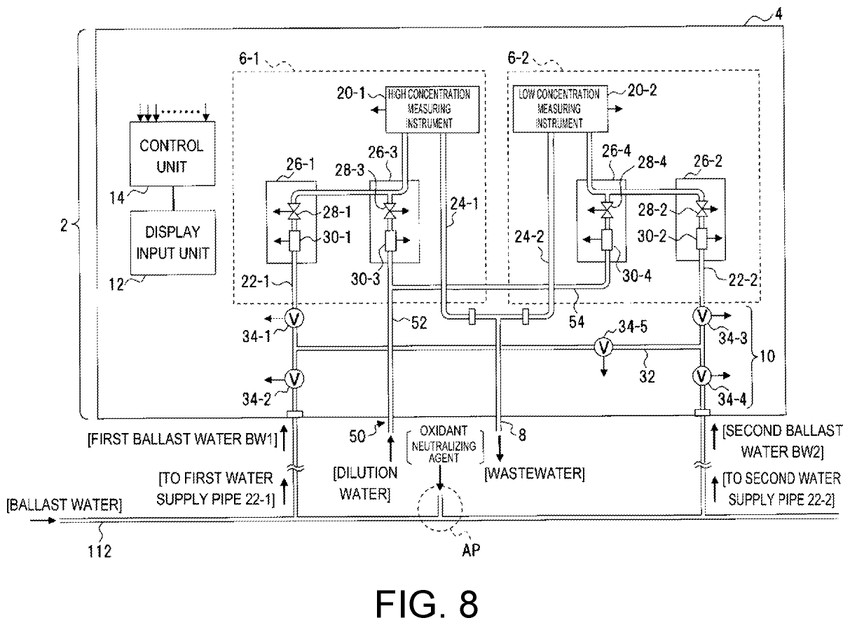

As illustrated in FIG. 8, the dilution water pipe 52 of the dilution water supplying unit 50 may include a branch pipe 54. This branch pipe 54 is connected to the water supply pipe 22-2 on the downstream side of an installation position of the flow rate adjusting unit 26-2. For this branch pipe 54, a fourth flow rate adjusting unit 26-4 (hereinafter referred to as “the flow rate adjusting unit 26-4”) similar to the flow rate adjusting unit 26-3 is installed. The dilution water supplying unit 50 illustrated in FIG. 8 supplies dilution water with a certain flow rate to ballast water which has been flow-rate-adjusted by the flow rate adjusting unit 26-2 to dilute the ballast water at a certain rate. By providing the dilution water supplying unit 50, it is possible to dilute ballast water supplied to the low concentration measuring instrument 20-2. Therefore, for example, it is possible to dilute ballast water after addition of the oxidant with dilution water to measure the ballast water by the low concentration measuring instrument 20-2, and it is possible to increase the degree of freedom of operation of the measurement apparatus 2. For example, the high concentration measuring instrument 20-1 can be temporarily suspended.

The dilution water supplying unit 50 may be used to supply flushing water for performing flushing (water-flow-type cleaning) of the high concentration measuring instrument 20-1 or the low concentration measuring instrument 20-2. By using the dilution water supplying unit 50 to supply the flushing water, a line dedicated for flushing can be omitted, and complicatedness and enlargement of the apparatus can be prevented.

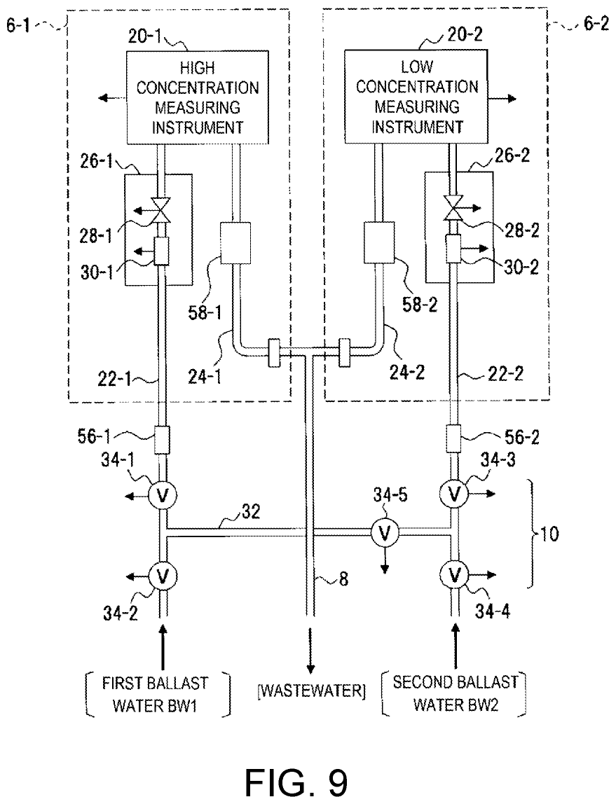

(6) As illustrated in FIG. 9, the water supply pipes 22-1 and 22-2 may be provided with pressure adjusting units 56-1 and 56-2 that adjust the pressure of ballast water. These pressure adjusting units 56-1 and 56-2 can be, for example, pressure adjusting valves. The pressure adjusting unit 56-1 installed in the water supply pipe 22-1 adjusts the pressure of ballast water in the water supply pipe 22-1, and the pressure adjusting unit 56-2 installed in the water supply pipe 22-2 adjusts the pressure of ballast water in the water supply pipe 22-2. The pressure adjusting units 56-1 and 56-2 control pressure fluctuation of the ballast waters in the water supply pipes 22-1 and 22-2, and it is possible to control influence on water quality measurement due to the pressure fluctuation of the ballast water. The pressure adjusting units 56-1 and 56-2 illustrated in FIG. 9 are installed in the water supply pipes 22-1 and 22-2 on the downstream side of the switching unit 10, but the pressure adjusting units 56-1 and 56-2 may be installed in the water supply pipes 22-1 and 22-2 on the upstream side of the switching unit 10.

The drainage pipes 24-1 and 24-2 may be provided with backflow preventing units 58-1 and 58-2 that prevent backflow of wastewater. These backflow preventing units 58-1 and 58-2 can be, for example, backflow prevention valves or on-off valves that open or close in response to a control signal of the control unit 14. The backflow preventing unit 58-1 installed in the drainage pipe 24-1 prevents wastewater of the measuring unit 6-2 from flowing into the measuring unit 6-1, and the backflow preventing unit 58-2 installed in the drainage pipe 24-2 prevents wastewater of the measuring unit 6-1 from flowing into the measuring unit 6-2. When either the measuring unit 6-1 or the measuring unit 6-2 is operating, it is prevented that the pressure of wastewater is added to the measuring unit 6-1 or the measuring unit 6-2 that is not operating, and it is also prevented that the measuring unit 6-1 or the measuring unit 6-2 is polluted with the wastewater.

(7) Though the display input unit 12 having a display function and an input function is provided in the above embodiment, a display unit and an input unit may be separately provided. Measurement apparatus information may be displayed on an external display device connected to the measurement apparatus 2. Instruction information generated by an external input device connected to the measurement apparatus 2 may be received.

(8) Though the measurement apparatus 2 is provided with the two measuring units 6-1 and 6-2 in the above embodiment, three or more measuring units may be provided. Even if the number of measuring units is three or more, the measuring units can be controlled by the control unit 14.

(9) Though the water quality information about the ballast water BW1 and the water quality information about the ballast water BW2 are displayed on the display screen 46 side by side in the above embodiment, the display method is not limited to such a display method. For example, if the water quality information about the ballast water BW1 and the water quality information about the ballast water BW2 are displayed on different screens, more water quality information about the ballast waters BW1 and BW2 can be displayed. If information including water quality, water quality transition and an alarm is displayed on one display screen, a burden of switching among displays is reduced.

(10) Though the measurement apparatus 2 is provided with the draining unit 8 in the above embodiment, wastewaters from the measuring units 6-1 and 6-2 may be separately discharged from the measurement apparatus 2.

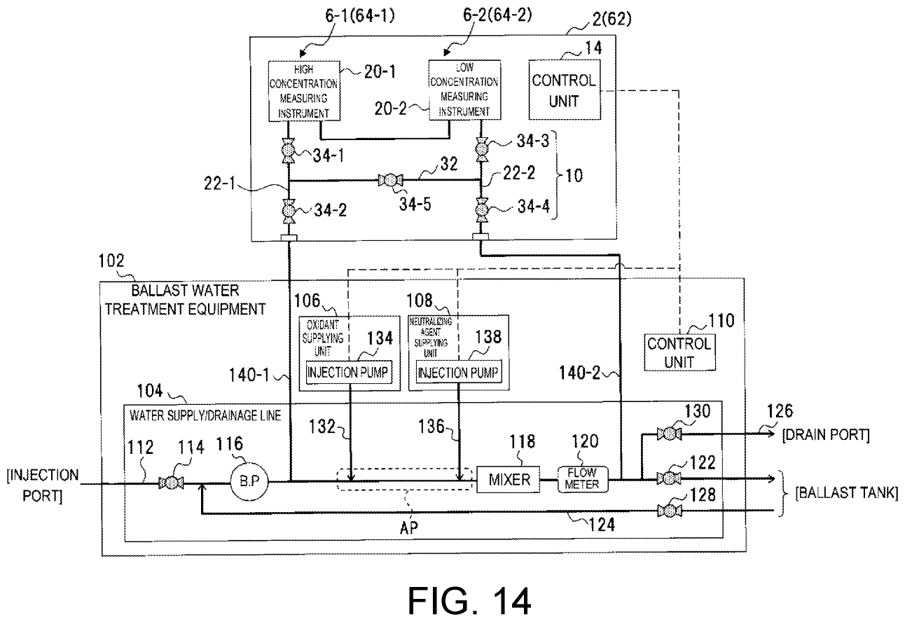

(11) The control unit 14 may output an instruction to adjust the amount of oxidant or neutralizing agent to be added, to an oxidant supplying unit 106 (FIG. 14) or a neutralizing agent supplying unit 108 (FIG. 14) of the ballast water treatment equipment according to a result of water quality measurement of ballast water. For example, at the time of the ballast water supply treatment, the control unit 14 may output a control signal to an oxidant injecting pump or oxidant injection controlling valve of the ballast water treatment equipment according to oxidant concentration information about ballast water after addition of the oxidant acquired from the measuring unit 6-1. Further, for example, at the time of the ballast water drainage treatment, the control unit 14 may output a control signal to a neutralizing agent injecting pump or neutralizing agent injection controlling valve of the ballast water treatment equipment according to oxidant concentration information about ballast water before neutralization, that is, the ballast water after addition of the oxidant acquired from the measuring unit 6-1. Further, for example, at the time of the ballast water drainage treatment, the control unit 14 may output a control signal to a neutralizing agent injecting pump or neutralizing agent injection controlling valve of the ballast water treatment equipment according to oxidant concentration information about ballast water after neutralization acquired from the measuring unit 6-2. By the control unit 14 of the measurement apparatus 2 directly controlling the pumps or the control valves, instruction routes for the pumps or the control valves are simplified.

(12) The control unit 14 may judge whether the oxidant or the neutralizing agent is excessively or insufficiently added, based on a result of water quality measurement of ballast water and output, for example, alarm information or a signal to stop (shut down) the ballast water treatment equipment. For example, if the oxidant concentration of ballast water exceeds a set value P [mg/L] a set number of times q, for example, three to five times in the ballast water supply treatment, the control unit 14 may output the alarm information or the signal to stop (shut down) the ballast water treatment equipment to stop the ballast water treatment equipment. By this alarm information outputting or equipment stopping process, excessive addition of the oxidant is prevented, and it is possible to control the amount of oxidant used to be a proper amount. Further, for example, if the oxidant concentration of ballast water after neutralization exceeds a set value K [mg/L] a set number of times n, for example, three to five times in the ballast water drainage treatment, the control unit 14 may output alarm information or a signal to stop the ballast water treatment equipment to stop the ballast water treatment equipment. By this alarm information outputting or equipment stopping process, it is prevented that insufficiently neutralized ballast water is drained into the sea, and it is possible to control the amount of oxidant discharged into the sea. Furthermore, judgment about insufficient addition of the oxidant may be omitted. Insufficient addition of the oxidant can be adjusted by adding the oxidant into a ballast tank. Though one measurement cycle can be arbitrarily set, about one minute to two minutes is preferred when proper control of the oxidant or neutralizing agent concentration is considered.

The set number of times q and the set value P only have to be set so that excessive addition of the oxidant is prevented. The set number of times n and the set value K only have to be set so that the amount of oxidant discharged is controlled. The set numbers of times and the set values are not limited to particular numbers of times and values. For example, the set number of times n may be reduced to a set number of times m, for example, two to three times, while the set value K may be doubled, that is, increased to a set value 2K. These set numbers of times q, n and m and the set values P, K and 2K can be set, for example, in the memory section 42 of the control unit 14.

(13) The control unit 14 may directly output one or more of a result of water quality measurement of ballast water, and information to stop the ballast water treatment equipment associated with output of the alarm information or a signal to stop the ballast water treatment equipment already described in the above modification, to a management apparatus on the ship side. The control unit 14 may output one or more of a result of water quality measurement of ballast water, and the alarm information or the information to stop the ballast water treatment equipment already described in the above modification, for example, to a management apparatus in a ship management center established on land through a communication line. It is possible for the management apparatus on the ship side or the management apparatus on land to grasp an alarm state or a stopped state of the ballast water treatment equipment, and it is possible to promote coping with the alarm state or the stopped state. Further, since the control unit 14 directly outputs the alarm or the stop information, a burden on a control unit on the ballast water treatment equipment side can be reduced.

### Second Embodiment

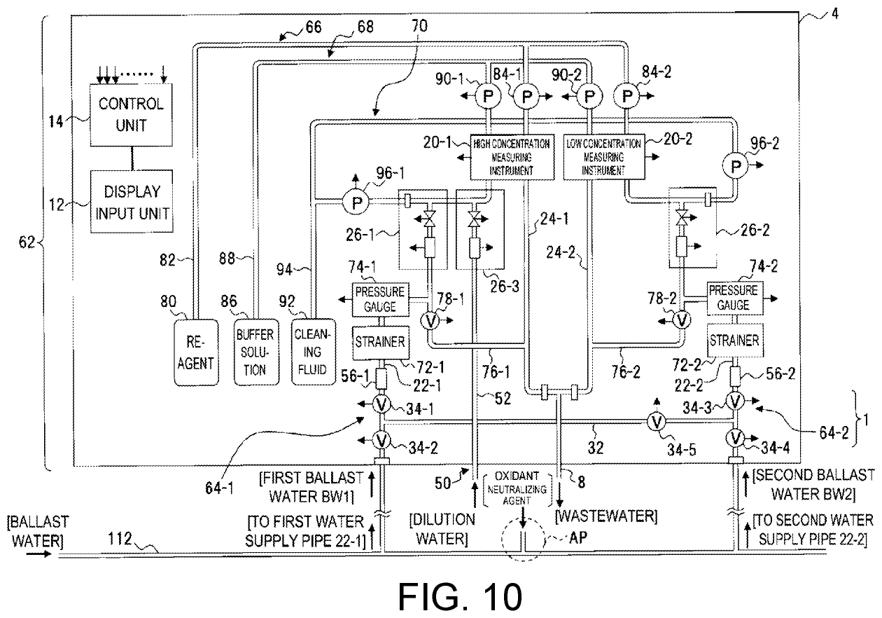

A second embodiment will be described with reference to FIG. 10. FIG. 10 illustrates an example of a ballast water measurement apparatus according to the second embodiment. In FIG. 10, the same parts as those in FIGS. 1, 7 and 9 are given the same reference numerals. In FIG. 10, thin arrows attached to devices, such as measuring instruments and flow rate adjusting units, and a control unit indicate connections between the control unit and the devices, and thick arrows attached to an inlet of a water supply pipe, an outlet of a draining unit, an inlet of a dilution water pipe and a water treatment line indicate directions in which ballast water, wastewater, dilution water, the oxidant or the neutralizing agent flows.

A ballast water measurement apparatus 62 (hereinafter referred to as “the measurement apparatus 62”) is an example of the measurement apparatus for measuring water qualities of a plurality kinds of ballast waters, for example, the already described oxidant concentrations. The measurement apparatus 62 is provided with the casing 4, the draining unit 8, the switching unit 10, the display input unit 12, the control unit 14 and the dilution water supplying unit 50 that have been already described in the first embodiment. Further, the measurement apparatus 62 is provided with a first measuring unit 64-1 (hereinafter referred to as “the measuring unit 64-1”), a second measuring unit 64-2 (hereinafter referred to as “the measuring unit 64-2”), a reagent supplying unit 66, a buffer solution supplying unit 68 and a cleaning fluid supplying unit 70.

The casing 4 accommodates the measuring unit 64-1, the measuring unit 64-2, the draining unit 8, the switching unit 10, the display input unit 12, the control unit 14, the dilution water supplying unit 50, the reagent supplying unit 66, the buffer solution supplying unit 68 and the cleaning fluid supplying unit 70, and these members are put together in the casing. This casing 4 is, for example, a metal casing and gives rigidity to the measurement apparatus 62.

The measuring unit 64-1 is provided with the high concentration measuring instrument 20-1, the water supply pipe 22-1, the drainage pipe 24-1, the flow rate adjusting unit 26-1 and the pressure adjusting unit 56-1. The high concentration measuring instrument 20-1, the water supply pipe 22-1, the drainage pipe 24-1, the flow rate adjusting unit 26-1 and the pressure adjusting unit 56-1 are similar to those of the first embodiment, and description thereof will be omitted. The measuring unit 64-2 is further provided with a first strainer 72-1 (hereinafter referred to as “the strainer 72-1”), a first pressure gauge 74-1 (hereinafter referred to as “the pressure gauge 74-1”) and a first bypass path 76-1 (hereinafter referred to as “the bypass path 76-1”).

The strainer 72-1 is installed in the water supply pipe 22-1 and functions as a filter for filtering floating matters in ballast water supplied to the measuring unit 64-1.

The pressure gauge 74-1 is installed in the water supply pipe 22-1. The pressure gauge 74-1 is arranged on the upstream side of the flow rate adjusting unit 26-1 and detects the inlet pressure of ballast water supplied to the measuring unit 64-1.

The bypass path 76-1 is connected to the water supply pipe 22-1 and the drainage pipe 24-1 to form a detour for the measuring unit 64-1. The bypass path 76-1 is provided with a bypass valve 78-1 to detour ballast water supplied to the measuring unit 64-1 when the bypass valve 78-1 is in an open state, and prevents the ballast water from being detoured when the bypass valve 78-1 is in a closed state.

The measuring unit 64-2 is provided with the low concentration measuring instrument 20-2, the water supply pipe 22-2, the drainage pipe 24-2, the flow rate adjusting unit 26-2 and the pressure adjusting unit 56-2. The low concentration measuring instrument 20-2, the water supply pipe 22-2, the drainage pipe 24-2, the flow rate adjusting unit 26-2 and the pressure adjusting unit 56-2 are similar to those of the first embodiment, and description thereof will be omitted. The measuring unit 64-2 is further provided with a second strainer 72-2 (hereinafter referred to as “the strainer 72-2”), a second pressure gauge 74-2 (hereinafter referred to as “the pressure gauge 74-2”) and a second bypass path 76-2 (hereinafter referred to as “the bypass path 76-2”).

The strainer 72-2 is installed in the water supply pipe 22-2 and functions as a filter for filtering floating matters in ballast water supplied to the measuring unit 64-2.

The pressure gauge 74-2 is installed in the water supply pipe 22-2. The pressure gauge 74-2 is arranged on the upstream side of the flow rate adjusting unit 26-2 and detects the inlet pressure of ballast water supplied to the measuring unit 64-2.

The bypass path 76-2 is connected to the water supply pipe 22-2 and the drainage pipe 24-2 to form a detour for the measuring unit 64-2. The bypass path 76-2 is provided with a bypass valve 78-2 to detour ballast water supplied to the measuring unit 64-2 when the bypass valve 78-2 is in an open state, and prevents the ballast water from being detoured when the bypass valve 78-2 is in a closed state.

The measuring units 64-1 and 64-2 are arranged, for example, symmetrically relative to an extension passing through the draining unit 8. By symmetrically arranging the measuring units 64-1 and 64-2, it is not necessary to separately grasp placements of the measuring units 64-1 and 64-2, so that a burden of handling the measurement apparatus 62 is reduced.

The reagent supplying unit 66 is provided with a reagent container 80, a reagent pipe 82, a first reagent supply pump 84-1 (hereinafter referred to as “the reagent supply pump 84-1”) and a second reagent supply pump 84-2 (hereinafter referred to as “the reagent supply pump 84-2”). The reagent container 80 is an example of a reagent storing section for storing a reagent. The reagent container 80 is connected, for example, to the high concentration measuring instrument 20-1 of the measuring unit 64-1 and the low concentration measuring instrument 20-2 of the measuring unit 64-2 via the reagent pipe 82. The reagent pipe 82 is an example of means for conveying the reagent. As for the reagent pipe 82, a pipe connected to the reagent container 80 is branched so that two branched pipes are formed. One of the branched pipes is connected to the high concentration measuring instrument 20-1, and the other branched pipe is connected to the low concentration measuring instrument 20-2. The reagent pipe 82 only has to be a pipe having chemical resistance against the reagent and is, for example, a resin pipe such as a fluororesin pipe or a vinyl chloride pipe, a stainless pipe, a corrosion-prevention-treated metal pipe or the like.

The reagent supply pump 84-1 is installed for the branched pipe connected to the high concentration measuring instrument 20-1. The reagent supply pump 84-1 supplies the reagent in the reagent container 80 to the high concentration measuring instrument 20-1 by being driven. The reagent supply pump 84-2 is installed for the branched pipe connected to the high concentration measuring instrument 20-2. The reagent supply pump 84-2 supplies the reagent in the reagent container 80 to the low concentration measuring instrument 20-2 by being driven. The reagent supply pumps 84-1 and 84-2 are, for example, metering pumps and supply a certain amount of reagent in response to supply of a certain amount of ballast water by the flow rate adjusting units 26-1 and 26-2 or supply of a certain amount of dilution water by the flow rate adjusting unit 26-3.

The reagent only has to react, for example, to TRO to be colored. For example, the already described DPD reagent can be used.

The buffer solution supplying unit 68 is provided with a buffer solution container 86, a buffer solution pipe 88, a first buffer solution supply pump 90-1 (hereinafter referred to as “the buffer solution supply pump 90-1”) and a second buffer solution supply pump 90-2 (hereinafter referred to as “the buffer solution supply pump 90-2”). The buffer solution container 86 is an example of a buffer solution storing section for storing a buffer solution. The buffer solution container 86 is connected, for example, to the high concentration measuring instrument 20-1 of the measuring unit 64-1 and the low concentration measuring instrument 20-2 of the measuring unit 64-2 via the buffer solution pipe 88. The buffer solution pipe 88 is an example of means for conveying the buffer solution. As for the buffer solution pipe 88, a pipe connected to the buffer solution container 86 is branched so that two branched pipes are formed. One of the branched pipes is connected to the high concentration measuring instrument 20-1, and the other branched pipe is connected to the low concentration measuring instrument 20-2. The buffer solution pipe 88 only has to be a pipe having chemical resistance against the buffer solution and is, for example, a resin pipe such as a fluororesin pipe or a vinyl chloride pipe, a stainless pipe, a corrosion-prevention-treated metal pipe or the like.

The buffer solution supply pump 90-1 is installed for the branched pipe connected to the high concentration measuring instrument 20-1. The buffer solution supply pump 90-1 supplies the buffer solution in the buffer solution container 86 to the high concentration measuring instrument 20-1 by being driven. The buffer solution supply pump 90-2 is installed for the branched pipe connected to the low concentration measuring instrument 20-2. The buffer solution supply pump 90-2 supplies the buffer solution in the buffer solution container 86 to the low concentration measuring instrument 20-2 by being driven. The buffer solution supply pumps 90-1 and 90-2 are, for example, metering pumps and supply a certain amount of buffer solution in response to supply of a certain amount of ballast water by the flow rate adjusting units 26-1 and 26-2 or supply of a certain amount of dilution water by the flow rate adjusting unit 26-3.

The buffer solution only has to be a solution for adjusting the hydrogen ion concentrations of the ballast waters BW1 and BW2 measured by the measuring instruments 20-1 and 20-2, and is, for example, a phosphate buffer solution.

The cleaning fluid supplying unit 70 is provided with a cleaning fluid container 92, a cleaning fluid pipe 94, a first cleaning fluid supply pump 96-1 (hereinafter referred to as “the cleaning fluid supply pump 96-1”) and a second cleaning fluid supply pump 96-2 (hereinafter referred to as “the cleaning fluid supply pump 96-2”) and cleans the high concentration measuring instrument 20-1 and the low concentration measuring instrument 20-2. The cleaning fluid container 92 is an example of a cleaning fluid storing section for storing a cleaning fluid. The cleaning fluid container 92 is connected to the water supply pipes 22-1 and 22-2 via the cleaning fluid pipe 94. The cleaning fluid pipe 94 is an example of means for conveying the cleaning fluid. As for the cleaning fluid pipe 94, a pipe connected to the cleaning fluid container 92 is branched so that two branched pipes are formed. One of the branched pipes is connected to the water supply pipe 22-1, and the other branched pipe is connected to the water supply pipe 22-2. The cleaning fluid pipe 94 only has to be a pipe that does not cause material change due to contact with water and is, for example, a resin pipe such as a fluororesin pipe or a vinyl chloride pipe, a stainless pipe, a corrosion-prevention-treated metal pipe or the like.

The cleaning fluid supply pump 96-1 is installed for the branched pipe connected to the water supply pipe 22-1. The cleaning fluid supply pump 96-1 supplies the cleaning fluid in the cleaning fluid container 92 to the water supply pipe 22-1 by being driven. The cleaning fluid supply pump 96-2 is installed for the branched pipe connected to the water supply pipe 22-2. The cleaning fluid supply pump 96-2 supplies the cleaning fluid in the cleaning fluid container 92 to the water supply pipe 22-2 by being driven.

The control unit 14 has the configuration already described in the first embodiment, and description thereof will be omitted. In addition to performing the information processing already described in the first embodiment, the processor 40 of the control unit 14 performs information processing such as an instruction to drive each, an instruction to open/close the bypass valves 78-1 and 78-2, acquisition of pressure information obtained by the pressure gauges 74-1 and 74-2, and processing based on the pressure information, for example, judgment of the water quality measurement of ballast water. In addition to wiredly or wirelessly connecting to the connected equipment already described in the first embodiment, the I/O 44 of the control unit 14 wiredly or wirelessly connects to connected equipment such as each pump, the bypass valves 78-1 and 78-2, and the pressure gauges 74-1 and 74-2. Since other components of the control unit 14 are similar to those of the control unit 14 of the first embodiment, description thereof will be omitted.

In this embodiment, since the dilution water supplying unit 50 is provided, ballast water supplied to the measuring unit 64-1 can be diluted by supplying dilution water. The amount of dilution water to be supplied can be adjusted by the control unit 14 so that the oxidant concentration of ballast water after dilution is within a favorable measurement range of the high concentration measuring instrument 20-1 (for example, 0.5 to 4.0 [mg/L] in chlorine equivalent). In the case of diluting ballast water, the control unit 14 can calculate the oxidant concentration of the ballast water in consideration of the amounts of ballast water and dilution water to be supplied. By diluting ballast water so that the oxidant concentration is within the favorable measurement range of the high concentration measuring instrument 20-1, the accuracy of measurement of the oxidant concentration can be increased.

[Water Quality Measurement of Ballast Waters BW1 and BW2]

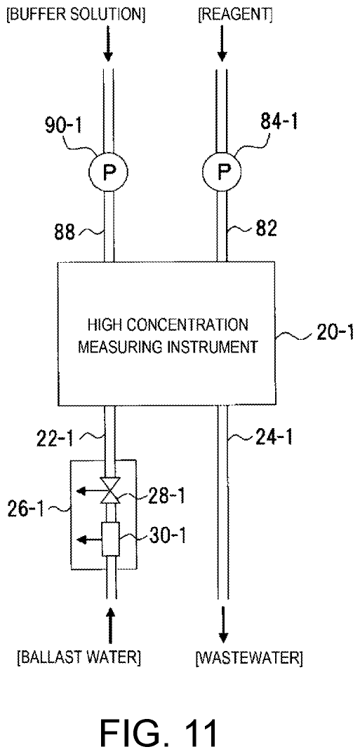

Next, FIG. 11 will be referred to about water quality measurement of the ballast waters BW1 and BW2. FIG. 11 illustrates the high concentration measuring instrument 20-1 and pipes connected to the high concentration measuring instrument 20-1. Arrows in FIG. 11 indicate flows of ballast water, wastewater, the reagent or the buffer solution. In FIG. 11, the dilution water supplying unit 50 is omitted.

The ballast water is supplied to the high concentration measuring instrument 20-1 via the water supply pipe 22-1 and the flow rate adjusting unit 26-1. The reagent is supplied to the high concentration measuring instrument 20-1 via the reagent pipe 82 and the reagent supply pump 84-1. The buffer solution is supplied to the high concentration measuring instrument 20-1 via the buffer solution pipe 88 and the buffer solution supply pump 90-1. The ballast water, the reagent and the buffer solution supplied to the high concentration measuring instrument 20-1 are mixed in the high concentration measuring instrument 20-1, for example, by a stirring device including a stirring chip and a stirrer. By the mixture of these, the reagent is caused to react to TRO in the ballast water and is colored into pink to pinkish red. The buffer solution adjusts the hydrogen ion concentration of the ballast water.

The high concentration measuring instrument 20-1 is provided with, for example, a colorimeter including a light source and measurement cells, measures light absorption strength of the ballast water colored according to a TRO concentration by addition of the reagent, and measures the TRO concentration of the ballast water including the DPD reagent and the buffer solution by the DPD colorimetric method or the DPD absorption spectrophotometry. The TRO concentration of the ballast water can be measured, for example, based on a DPD colorimetric method described in item 33.2 of or a DPD absorption spectrophotometry described in item 33.4 of Japanese Industrial Standards JIS K0102 (2013), or DPD Colorimetric Method 4500-C1 G approved by United States Environmental Protection Agency, or the like.

The ballast water after water quality measurement is discharged via the drainage pipe 24-1 as wastewater. The water quality of ballast water supplied to the low concentration measuring instrument 20-2 can be measured similarly to the water quality of ballast water supplied to the high concentration measuring instrument 20-1, using the low concentration measuring instrument 20-2, the water supply pipe 22-2, the flow rate adjusting unit 26-2, the reagent pipe 82, the reagent supply pump 84-2, the buffer solution pipe 88, the buffer solution supply pump 90-2 and the drainage pipe 24-2. Description of water quality measurement of the ballast water supplied to the low concentration measuring instrument 20-2 will be omitted.

[Process Procedure for Water Quality Measurement of Ballast Waters BW1 and BW2]

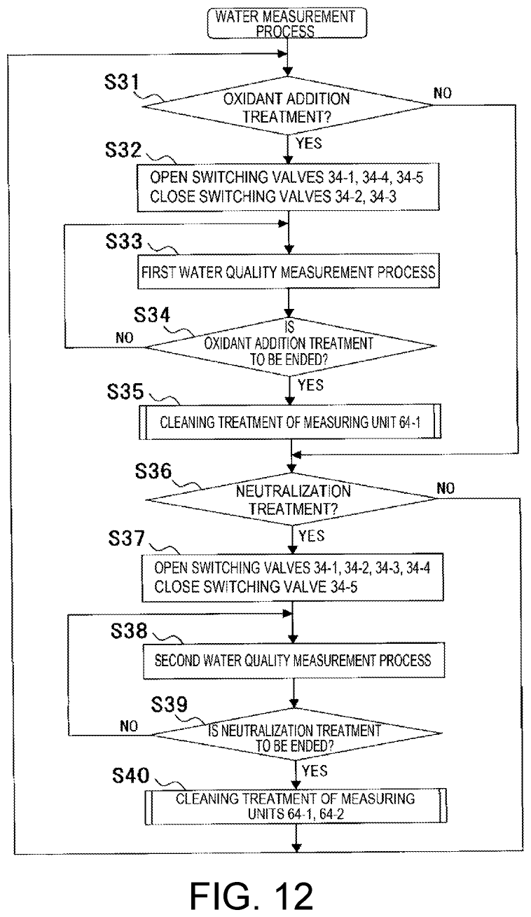

Next, FIG. 12 will be referred to about a process procedure for water quality measurement of the ballast waters BW1 and BW2. FIG. 12 is a flowchart illustrating an example of a water quality measurement process procedure. This water quality measurement process procedure is an example of a ballast water measurement method of the present invention and is performed by the control unit 14. In FIG. 12, step S indicates a process stage.

The control unit 14 judges whether the oxidant addition treatment is to be performed (step S31). For example, the control unit 14 can acquire ballast water treatment information indicating the ballast water supply treatment already described in the first embodiment and judge that the oxidant addition treatment is to be performed. If the oxidant addition treatment is to be performed (step S31: YES), the control unit 14 opens the switching valves 34-1, 34-4 and 34-5 and closes the switching valves 34-2 and 34-3 (step S32). After that, the control unit 14 performs a first water quality measurement process (step S33). This first water quality measurement process is an example of the water quality measurement process in the oxidant addition treatment. In this first water quality measurement process, the control unit 14 causes the reagent supply pump 84-1 and the buffer solution supply pump 90-1 to operate to supply the reagent and the buffer solution to the high concentration measuring instrument 20-1. Other parts of the first water quality measurement process is similar to those of the first water quality measurement process (steps S13 to S15) already described in the first embodiment, and description thereof will be omitted. The control unit 14 may further cause the flow rate adjusting unit 26-3 to operate to supply dilution water to ballast water or monitor the pressure of the ballast water based on a pressure detected by the pressure gauge 74-1.

The control unit 14 judges whether the oxidant addition treatment is to be ended (step S34). If the oxidant addition treatment is not to be ended (step S34: NO), the control unit 14 returns to step S33 and repeats the first water quality measurement process (step S33) and step S34. The control unit 14 can judge end of the oxidant addition treatment by cease of the ballast water treatment information indicating the ballast water supply treatment or by acquisition of ballast water treatment information indicating end of the ballast water supply treatment from the ballast water treatment equipment.

If the oxidant addition treatment is to be ended (step S34: YES), cleaning treatment of the measuring unit 64-1 is performed (step S35). If the oxidant addition treatment is not to be performed (step S31: NO), steps S32 to S35 are omitted.

If the oxidant addition treatment is not to be performed (step S31: NO) or after the cleaning treatment of the measuring unit 64-1 (step S35), the control unit 14 judges whether the neutralization treatment is to be performed (step S36). For example, the control unit 14 can acquire ballast water treatment information indicating the ballast water drainage treatment already described in the first embodiment and judge that the neutralization treatment is to be performed. If the neutralization treatment is to be performed (step S36: YES), the control unit 14 opens the switching valves 34-1, 34-2, 34-3 and 34-4 and closes the switching valve 34-5 (step S37). After that, the control unit 14 performs a second water quality measurement process (step S38). This second water quality measurement process is an example of the water quality measurement process in the neutralization treatment. In this second water quality measurement process, the control unit 14 causes the reagent supply pumps 84-1 and 84-2, and the buffer solution supply pumps 90-1 and 90-2 to operate to supply the reagent and the buffer solution to the measuring instruments 20-1 and 20-2. Other parts of the second water quality measurement process is similar to those of the second water quality measurement process (steps S19 to S21) already described in the first embodiment, and description thereof will be omitted. The control unit 14 may further cause the flow rate adjusting unit 26-3 to operate to supply dilution water to the ballast water or monitor the pressure of the ballast water based on detected pressures of the pressure gauges 74-1 and 74-2.

The control unit 14 judges whether the neutralization treatment is to be ended (step S39). If the neutralization treatment is not to be ended (step S39: NO), the control unit 14 returns to step S38 and repeats the second water quality measurement process (step S38) and step S39. The control unit 14 can judge end of the neutralization treatment by cease of ballast water treatment information indicating the ballast water drainage treatment or by acquisition of ballast water treatment information indicating end of the ballast water drainage treatment from the ballast water treatment equipment.

If the neutralization treatment is to be ended (step S39: YES), the cleaning treatment of the measuring units 64-1 and 64-2 is performed (step S40). If the neutralization treatment is not to be performed (step S36: NO), steps S37 to S40 are omitted.

The control unit 14 can repeat this process procedure and continuously or intermittently measure the water quality of ballast water. Since the already described first quality measurement process and second quality measurement process are individually performed, the control unit 14 can not only perform both of the first water quality measurement process and the second water quality measurement process but also perform either the first water quality measurement process or the second water quality measurement process.

[Cleaning Treatment of Measuring Units 64-1 and 64-2]

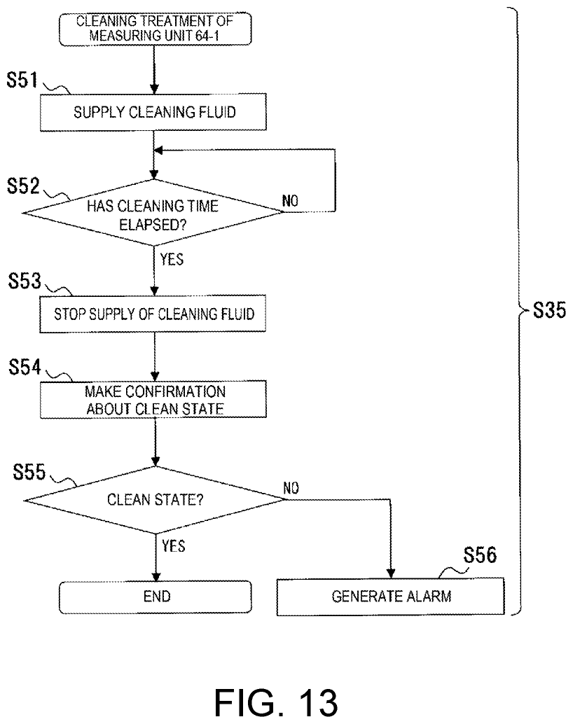

Next, FIG. 13 will be referred to about the cleaning treatment (step S35) of the measuring unit 64-1. FIG. 13 illustrates an example of a process procedure for cleaning treatment of the measuring unit. This process procedure for cleaning treatment of the measuring unit is a subroutine process in the cleaning treatment (step S35) of the measuring unit 64-1. In FIG. 13, step S indicates a process stage.

In this cleaning treatment of the measuring unit 64-1, the control unit 14 causes the cleaning fluid supply pump 96-1 to operate to supply cleaning fluid in the cleaning fluid container 92 to the water supply pipe 22-1 (step S51). The cleaning fluid supplied to the water supply pipe 22-1 passes through the water supply pipe 22-1 and the high concentration measuring instrument 20-1 to clean these, and is discharged outside the measurement apparatus 62 via the drainage pipe 24-1 and the draining unit 8.

After start of supply of the cleaning fluid, it is judged whether cleaning time set in advance has elapsed (step S52). If the cleaning time has not elapsed (step S52: NO), step S52 is repeated until the cleaning time elapses. This cleaning time can be, for example, time required to clean the water supply pipe 22-1 and the high concentration measuring instrument 20-1. The set cleaning time is set, for example, in the memory section 42 of the control unit 14. If the cleaning time elapses (step S52: YES), supply of the cleaning fluid is stopped (step S53), and whether the high concentration measuring instrument 20-1 of the measuring unit 64-1 is in a clean state is confirmed (step S54). This conformation about the clean state can be performed, for example, by the control unit 14 acquiring a measurement value from the cleaned high concentration measuring instrument 20-1 and judging whether the measurement value is a normal value. If the high concentration measuring instrument 20-1 is in the clean state (step S55: YES), the cleaning treatment is ended. If the high concentration measuring instrument 20-1 is not in the clean state (step S55: NO), that is, the high concentration measuring instrument 20-1 is insufficiently cleaned, the control unit 14 generates an alarm (step S56) and stops the cleaning treatment.

In the cleaning treatment of the measuring units 64-1 and 64-2 (step S40), cleaning treatment of the measuring unit 64-2 is also performed in addition to the already described cleaning treatment of the measuring unit 64-1. The cleaning treatment of the measuring unit 64-2 can be performed similarly to the cleaning treatment of the measuring unit 64-1 (step S35), and description thereof will be omitted.

[Advantageous Effects of Second Embodiment]

(1) The advantageous effects already described in the first embodiment can be obtained.

(2) Since the measurement apparatus 62 is provided with the dilution water supplying unit 50, it is possible to, for example, in order that the oxidant concentration of ballast water supplied to the high concentration measuring instrument 20-1 is within a favorable measurement range for the high concentration measuring instrument 20-1, dilute the ballast water, and it is possible to increase the accuracy of measurement of the oxidant concentration.

(3) Since the water supply pipes 22-1 and 22-2 are provided with the pressure adjusting units 56-1 and 56-2 for adjusting the pressure of ballast water, pressure fluctuation of ballast waters in the water supply pipes 22-1 and 22-2 is controlled, and it is possible to control influence on water quality measurement due to the pressure fluctuation of the ballast water.

(4) Since the measurement apparatus 62 is provided with the cleaning fluid supplying unit 70, the clean state of the measuring instruments 20-1 and 20-2 is maintained, and it is prevented that an abnormality such as a measurement abnormality occurs due to dirt on the measuring instruments 20-1 and 20-2. That is, since the clean state of the measuring instruments 20-1 and 20-2 can be monitored, it is prevented that a measurement abnormality due to dirt on the measuring instruments 20-1 and 20-2 is neglected, and measurement is repeated in the state of measurement abnormality.

(5) Since each of the number of reagent containers 80, the number of buffer solution containers 80 and the number of cleaning fluid container 92 is smaller than the number of positions for water quality measurement of ballast water, a burden of equipment management of the measurement apparatus 62 and a burden of management of remaining amounts of reagent and buffer solution are reduced. Further, since supply of the reagent, the buffer solution and the cleaning fluid is shared by the two measuring units 64-1 and 64-2, an area required for installation of the reagent supplying unit 66, the buffer solution supplying unit 68 and the cleaning fluid supplying unit 70 is reduced. Therefore, it is possible to reduce an area for installing the measurement apparatus 62 and reduce a work area required for operation or maintenance of the measurement apparatus 62.

(6) In the cleaning treatment of the measuring units 64-1 and 64-2 in the above embodiment, if the measuring instruments 20-1 and 20-2 of the measuring units 64-1 and 64-2 are not in clean state, that is, if they are insufficiently cleaned, an alarm is generated to notify an abnormality. By this notification of the abnormality, water quality measurement using the measuring instruments 20-1 and 20-2 in an insufficiently cleaned state is prevented, and it is possible to prevent continuation of measurement under an abnormal state. Since measurement under an abnormal state is prevented, it is possible to maintain proper injection of the reagent as well as a high accuracy of water quality measurement, and it is possible to prevent increase in reagent cost due to excessive injection of the reagent.

[Modification]

(1) The modification already described in the first embodiment can be applied to the second embodiment.

(2) Though the measuring unit 64-1 of the measurement apparatus 62 is provided with the pressure adjusting unit 56-1, the strainer 72-1, the pressure gauge 74-1 and the bypass path 76-1 in the above embodiment, these may be selectively provided. Though the measuring unit 64-2 of the measurement apparatus 62 is provided with the pressure adjusting unit 56-2, the strainer 72-2, the pressure gauge 74-2 and the bypass path 76-2, these may be selectively provided. In these modifications, the advantageous effects already described in the first embodiment can also be obtained.

(3) Though the above embodiment includes the cleaning treatment of the measuring units 64-1 and 64-2, and the measuring units 64-1 and 64-2 are cleaned when measurement of the ballast waters BW1 and BW2 ends, switching may be performed to clean the measuring units 64-1 and 64-2 during measurement of the ballast waters BW1 and BW2, and the measuring units 64-1 and 64-2 may be cleaned while measurement of the ballast waters BW1 and BW2 is stopped. Further, cleaning may be started, being triggered by detection of an abnormal value of the TRO concentration; cleaning may be started when dirt or insufficient cleaning of measurement cells that the measuring instruments 20-1 and 20-2 are provided is detected; or cleaning may be started based on time elapsed after the previous cleaning treatment.

(4) In the above embodiment, the cleaning fluid supplying unit 70 is connected to the water supply pipes 22-1 and 22-2, and the measuring instruments 20-1 and 20-2, and the water supply pipes 22-1 and 22-2 are cleaned by supply of cleaning fluid so that it is prevented that an abnormality occurs in a measurement result due to dirt on the measuring instruments 20-1 and 20-2 and the water supply pipes 22-1 and 22-2. However, instead of cleaning of the measuring instruments 20-1 and 20-2 and the water supply pipes 22-1 and 22-2 by the cleaning fluid supplying unit 70, the flushing (water-flow-type cleaning) by the dilution water supplying unit 50 already described in the first embodiment may be performed.

(5) Though the oxidant concentration of ballast water is measured by supplying the DPD reagent, the measurement is not limited to the measurement using the DPD reagent. For example, potassium iodide may be used as the reagent so that the oxidant concentration is measured using iodine generated by the potassium iodide reacting to TRO. Other water quality elements including TRO may be measured.

(6) The reagent used for water quality measurement of ballast water may be colored, for example, in blue in an initial state. When the colored reagent is used, ballast water including the reagent shows coloration due to reaction between the reagent and TRO and has the color that the reagent has had since the initial state. By measuring coloration of the reagent and the coloration in the initial state by the measuring instruments 20-1 and 20-2, the control unit 14 can detect a reagent empty state as well as the oxidant concentration. When the reagent empty state occurs, the control unit 14 can temporarily stop ballast water treatment on the ballast water treatment equipment side by outputting empty state information to the ballast water treatment equipment. By outputting the empty state information to the ballast water treatment equipment, it is possible to prevent a malfunction due to lack of the reagent, for example, prevention of injection of the neutralizing agent based on a misjudgment that the neutralizing agent is excessively injected. That is, drainage of insufficiently neutralized ballast water is prevented.

If the control unit 14 notifies the reagent empty state, a trigger for adding the reagent can be provided. A sailor only has to add the reagent based on the notification of the empty state and does not have to monitor the remaining amount of reagent in order not to run out of the reagent. That is, a burden on the sailor is reduced. Further, if the land-side management center is notified of reagent empty state information, for example, via a satellite line, the management center can recognize lack of the reagent. Since this notification of the empty state information is automatically performed by the control unit 14 of the measurement apparatus 62 without intervention of a sailor, notification omission is prevented. The management center that receives the empty state information can instruct the ship to take appropriate measures, and it is possible to shorten time to cope with deficiencies and improve treatment quality of ballast water treatment.

### Third Embodiment

A third embodiment will be described with reference to FIG. 14. FIG. 14 illustrates an example of connection between the ballast water measurement apparatus and ballast water treatment equipment. In FIG. 14, the same parts as those in FIG. 1 or 10 are given the same reference numerals. In this embodiment, the measurement apparatus 2 already described in the first embodiment may be connected to ballast water treatment equipment 102, or the measurement apparatus 62 already described in the second embodiment may be connected to the ballast water treatment equipment 102. The measurement apparatus 2 (62) illustrated in FIG. 14 and the ballast water treatment equipment 102 are shown as examples, and the present invention is not limited to the configuration. The measurement apparatus 2 (62) and the ballast water treatment equipment 102 are provided for a ship.

[Ballast Water Treatment Equipment 102]

The ballast water treatment equipment 102 is provided with a water supply/drainage line 104, the oxidant supplying unit 106, the neutralizing agent supplying unit 108 and a control unit 110.

The water supply/drainage line 104 is provided with the water treatment line 112, and this water treatment line 112 is provided with a ballast water injection valve 114, a ballast pump 116, a mixer 118, a flow meter 120 and a ballast tank inlet valve 122. The water treatment line 112 connects a ballast water injection port and the ballast tank. The ballast water injection valve 114 is installed on the downstream side of the injection port, and the ballast water injection valve 114 causes ballast water supplied from the injection port to pass through or stops the ballast water by opening or closing. The ballast pump 116 is installed on the downstream side of the ballast water injection valve 114, and flows the ballast water into the mixer 118 by being driven. The mixer 118 is installed on the downstream side of the ballast pump 116 and mixes the oxidant or the neutralizing agent injected between the ballast pump 116 and the mixer 118 with the ballast water. The flow meter 120 is installed on the downstream side of the mixer 118 and measures the flow rate of the ballast water including the oxidant or the neutralizing agent. The ballast tank inlet valve 122 is installed on the downstream side of the flow meter 120. The ballast tank inlet valve 122 causes the ballast water to pass through or prevents passage of the ballast water by opening or closing. The ballast water that has passed through the ballast tank inlet valve 122 is poured into the ballast tank.

The water supply/drainage line 104 is further provided with a branch line 124 and a drainage line 126 provided in parallel to the water treatment line 112. This branch line 124 is provided with a ballast tank outlet valve 128, and the drainage line 126 is provided with a ballast drainage valve 130. The branch line 124 connects the water treatment line 112 and the ballast tank between the ballast water injection valve 114 and the ballast pump 116. The drainage line 126 connects the water treatment line 112 and a drain port between the flow meter 120 and the ballast tank inlet valve 122. The ballast tank outlet valve 128 causes ballast water in the branch line 124 to pass through or prevents passage of the ballast water by opening or closing. The ballast drainage valve 130 causes ballast water in the drainage line 126 to pass through or prevents passage of the ballast water by opening or closing.

When the ballast water injection valve 114 and the ballast tank inlet valve 122 are opened, and the ballast tank outlet valve 128 and the ballast drainage valve 130 are closed, a ballast water supply route is formed. When the ballast pump 116 is caused to operate, ballast water passes through the water treatment line 112 and is supplied to the ballast tank.

When the ballast water injection valve 114 and the ballast tank inlet valve 122 are closed, and the ballast tank outlet valve 128 and the ballast drainage valve 130 are opened, a ballast water drainage route is formed. When the ballast pump 116 is caused to operate, the ballast water in the ballast tank flows through the branch line 124, the ballast pump 116, the mixer 118 and the flow meter 120 in that order and is drained from the drain port to the outside of the ship through the drainage line 126.

The oxidant supplying unit 106 is connected to the water treatment line 112 on the upstream side of the mixer 118 via an oxidant supply line 132 and supplies the oxidant by driving of an injection pump 134.

The neutralizing agent supplying unit 108 is connected to the water treatment line 112 on the upstream side of the mixer 118 via a neutralizing agent supply line 136 and supplies the neutralizing agent by operation of an injection pump 138. This neutralizing agent is, for example, sodium sulfite, sodium bisulfate (sodium hydrogen sulfite) or sodium thiosulfate. Connecting sections between the oxidant supply line 132 and the neutralizing agent supply line 136, and the water treatment line 112 form the chemical addition position AP already described in the first embodiment.

The control unit 110 is connected to each valve, the oxidant supplying unit 106, the neutralizing agent supplying unit 108, the flow meter 120 and the control unit 14 of the measurement apparatus 2 (or the measurement apparatus 62). The control unit 110 controls opening/closing of each valve and amounts of oxidant and neutralizing agent to be supplied, and judges or records a treatment state of ballast water in response to a measurement value of the flow meter 120 and water quality information about the ballast water. Furthermore, the control unit 110 can be connected to the ballast pump 116 to control operation or stop of the ballast pump 116 by the control unit 110.

[Connection Between Measurement Apparatus 2 and Ballast Water Treatment Equipment 102]

The water supply pipe 22-1 of the measurement apparatus 2 is connected to the water treatment line 112 on the upstream side of the oxidant supply line 132 and the neutralizing agent supply line 136 via a connection pipe 140-1, and the water supply pipe 22-2 of the measurement apparatus 2 is connected to the water treatment line 112 on the downstream side of the mixer 118 via a connection pipe 140-2. The two water supply pipes 22-1 and 22-2 of the measurement apparatus 2 are connected to the water treatment line 112 of the ballast water treatment equipment 102 by such connections, and the ballast water BW1 before the oxidant or the neutralizing agent being supplied is supplied to the water supply pipe 22-1, and the ballast water BW2 after the oxidant or the neutralizing agent being supplied is supplied to the water supply pipe 22-2. The ballast water BW1 supplied to the water supply pipe 22-1 is supplied to the high concentration measuring instrument 20-1 or the low concentration measuring instrument 20-2 by the switching unit 10, and the ballast water BW2 supplied to the water supply pipe 22-2 is supplied to the high concentration measuring instrument 20-1 or the low concentration measuring instrument 20-2 by the switching unit 10. Supply destinations of the ballast waters BW1 and BW2 can be changed by the switching unit 10, and it is possible to increase the accuracy of measurement of the oxidant concentration of ballast water.

[Treatment Sequence of Measurement Apparatus 2 and Ballast Water Treatment Equipment 102]

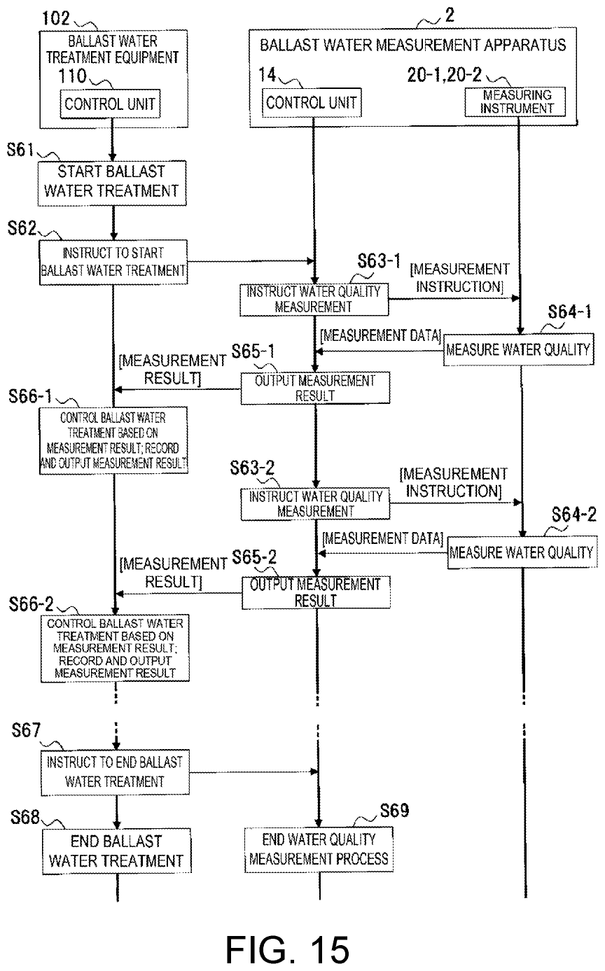

FIG. 15 illustrates an example of a treatment sequence of the ballast water measurement apparatus and the ballast water treatment equipment. In FIG. 15, step S indicates a process stage.

When the control unit 110 of the ballast water treatment equipment 102 starts ballast water treatment, that is, the ballast water supply treatment or the ballast water drainage treatment (step S61), the control unit 110 instructs the control unit 14 of the measurement apparatus 2 to start the ballast water treatment (step S62). In response to the instruction to start the ballast water treatment, the control unit 14 performs the water quality measurement process according to steps S12 to S16 of the water quality measurement process procedure (FIG. 4) already described in the first embodiment if the instruction is an instruction to start the ballast water supply treatment, and performs the water quality measurement process according to steps S18 to S22 of the water quality measurement process procedure (FIG. 4) if the instruction is an instruction to start the ballast water drainage treatment. In these water quality measurement processes, for example, the high concentration measuring instrument 20-1, or the measuring instruments 20-1 and 20-2 are instructed to perform water quality measurement according to whether the ballast water supply treatment or the ballast water drainage treatment (step S63-1), and the high concentration measuring instrument 20-1, or the measuring instruments 20-1 and 20-2 measure water quality and output measurement data to the control unit 14 (step S64-1). Receiving the water quality measurement data, the control unit 14 outputs a ballast water measurement result to the control unit 110 (step S65-1). The control unit 110 controls the ballast water treatment based on the measurement result, records the measurement result and outputs the measurement result to the ship side (step S66-1). Steps S63-1 to S66-1 are repeated until the ballast water treatment ends (step S63-2, step S64-2, step S65-2, step S66-2, . . . ). The control unit 110 instructs the control unit 14 to end the ballast water treatment (step S67) to end the ballast water treatment (step S68). In response to the instruction to end the ballast water treatment, the control unit 14 ends the water quality measurement process (step S69).

[Connection Between Measurement Apparatus 62 and Ballast Water Treatment Equipment 102, and Processing Sequence of Measurement Apparatus 62 and Ballast Water Treatment Equipment 102]

The measurement apparatus 62 and the ballast water treatment equipment 102 can be connected similarly to the measurement apparatus 2 and the ballast water treatment equipment 102. Further, the processing sequence of the measurement apparatus 2 and the ballast water treatment equipment 102 can be applied to the measurement apparatus 62 and the ballast water treatment equipment 102. When either the measuring units 6-1 and 64-1 or the measuring units 6-2 and 64-2 of the measurement apparatus 2 (62) are malfunctioning, it is possible to switch connections between the measuring units 6-1, 6-2, 64-1 and 64-2, and the water treatment line 112 by a switching mechanism to measure ballast water only by either the measuring units 6-1 and 64-1 or the measuring units 6-2 and 64-2, for example, so that ballast water in which neutralizing agent is insufficiently injected is prevented from being discharged.

[Advantageous Effects of Third Embodiment]

By connecting the two water supply pipes 22-1 and 22-2 of the measurement apparatus 2 or the measurement apparatus 62 to the connection pipes 140-1 and 140-2 of the ballast water treatment equipment 102 installed in the ship, the oxidant concentration of ballast water before and after ballast water treatment by the ballast water treatment equipment 102 can be measured by one measurement apparatus 2 or measurement apparatus 62. Therefore, it is possible to reduce an area for installing the measurement apparatus 2 or the measurement apparatus 62 and reduce a work area required for operation or maintenance of the measurement apparatus 2 or the measurement apparatus 62. The ballast water treatment equipment 102 can perform ballast water treatment and water quality management of ballast water by being linked with one measurement apparatus 2 or measurement apparatus 62, and it is easy to link the ballast water treatment equipment 102 with the measurement apparatus 2 or 62. Further, a burden of equipment management for the measurement apparatus 2 (62) and a burden of management of remaining amounts of reagent and buffer solution are reduced. With regard to water quality measurement of ballast water, a sailor does not have to maintain or manage a plurality of measurement apparatuses, and an equipment management burden on the sailor is reduced.

[Modification]

The oxidant concentration of ballast water may be measured for the ballast water before and after ballast water treatment or may be measured for the ballast water either before or after the ballast water treatment. In water quality measurement of the ballast water before the ballast water treatment, the ballast water treatment can be controlled by feedforward of a measurement result. In water quality measurement of the ballast water after the ballast water treatment, the ballast water treatment can be controlled by feedback of a measurement result, and the water quality of the ballast water after the ballast water processing can be actually measured.

### OTHER EMBODIMENTS

In the third embodiment, the control unit 14 of the ballast water measurement apparatus 2 (62) notifies the control unit 110 of the ballast water treatment equipment 102 of a measurement result of water quality measurement. However, as already described in the first embodiment, the control unit 14 may output an instruction to adjust the amount of oxidant or neutralizing agent to be added, according to the water quality measurement result of ballast water. The control unit 14 may judge whether the oxidant or the neutralizing agent is excessively or insufficiently added, based on a result of water quality measurement of ballast water and output, for example, alarm information or a signal to stop (shut down) the ballast water treatment equipment 102. The control unit 14 may directly output one or more of a result of water quality measurement of ballast water, and information to stop the ballast water treatment equipment 102 associated with output of the alarm information or the stop signal, to the management apparatus on the ship side, or output them to the management apparatus in the ship management center established on land through the communication line.

[Treatment Sequence of Measurement Apparatus 2 and Ballast Water Treatment Equipment 102]

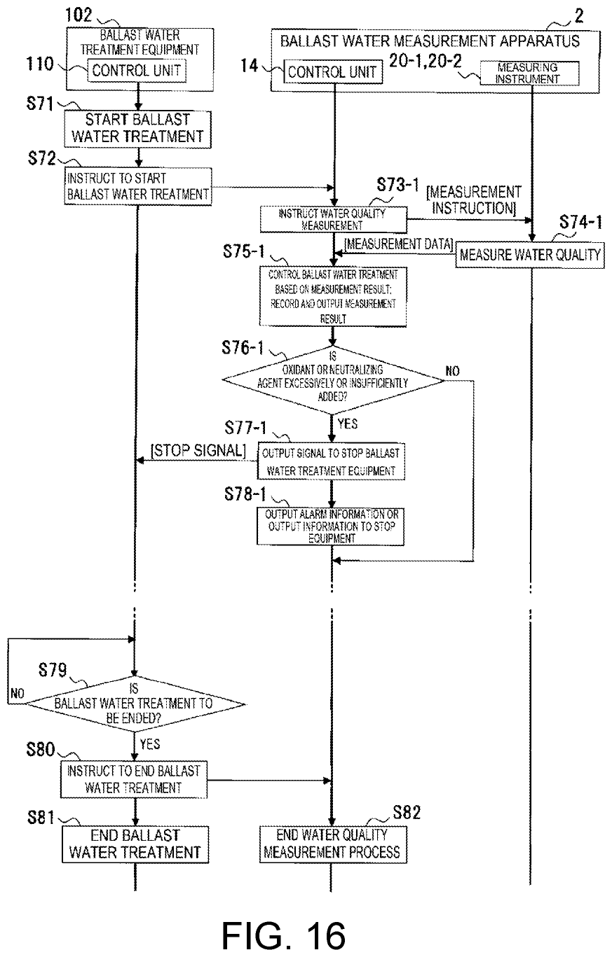

FIG. 16 illustrates an example of the treatment sequence of the ballast water measurement apparatus and the ballast water treatment equipment. In FIG. 16, step S indicates a process stage. In FIG. 16, the same parts as those in FIG. 15 are given the same reference numerals.

When the control unit 110 of the ballast water treatment equipment 102 starts ballast water treatment, that is, the ballast water supply treatment or the ballast water drainage treatment (step S71), the control unit 110 instructs the control unit 14 of the measurement apparatus 2 to start the ballast water treatment (step S72). In response to the instruction to start the ballast water treatment, the control unit 14 performs the water quality measurement process according to steps S12 to S16 of the water quality measurement process procedure (FIG. 4) already described in the first embodiment if the instruction is an instruction to start the ballast water supply treatment, and performs the water quality measurement process according to steps S18 to S22 of the water quality measurement process procedure (FIG. 4) if the instruction is an instruction to start the ballast water drainage treatment. In these water quality measurement processes, for example, the high concentration measuring instrument 20-1, or the measuring instruments 20-1 and 20-2 are instructed to perform water quality measurement according to whether the ballast water supply treatment or the ballast water drainage treatment (step S73-1), and the high concentration measuring instrument 20-1, or the measuring instruments 20-1 and 20-2 measure water quality and output measurement data to the control unit 14 (step S74-1). Receiving the water quality measurement data, the control unit 14 controls the ballast water treatment based on the measurement result, records the measurement result and outputs the measurement result to the ship side or the land side (step S75-1). Next, the control unit 14 judges whether the oxidant or the neutralizing agent is excessively or insufficiently added (step S76-1). If the oxidant or the neutralizing agent is excessively or insufficiently added (step S76-1: YES), the control unit 14 outputs a signal to stop the ballast water treatment equipment to the control unit 110 (step S77-1) and outputs alarm information or information to stop equipment to the ship side or the land side (step S78-1). If the oxidant or the neutralizing agent is not excessively or insufficiently added, that is, the amount of oxidant or neutralizing agent added is proper (step S76-1: NO), steps S77-1 and S78-1 are omitted. Excessive or insufficient addition of the oxidant or the neutralizing agent can be judged based on the set number of times q, n or m, and the set value P, K or 2K [mg/L] already described in the first embodiment.

Steps S73-1 to S78-1 are repeated until the ballast water treatment ends. The control unit 110 judges whether the ballast water treatment is to be ended (step S79). If the ballast water treatment is to be ended (step S79: YES), the control unit 110 instructs the control unit 14 to end the ballast water treatment (step S80) to end the ballast water treatment (step S81). In response to the instruction to end the ballast water treatment, the control unit 14 ends the water quality measurement process (step S82).

[Connection Between Measurement Apparatus 62 and Ballast Water Treatment Equipment 102, and Processing Sequence of Measurement Apparatus 62 and Ballast Water Treatment Equipment 102]

The measurement apparatus 62 and the ballast water treatment equipment 102 can be connected similarly to the measurement apparatus 2 and the ballast water treatment equipment 102. Further, the processing sequence of the measurement apparatus 2 and the ballast water treatment equipment 102 can be applied to the measurement apparatus 62 and the ballast water treatment equipment 102. When either the measuring units 6-1 and 64-1 or the measuring units 6-2 and 64-2 of the measurement apparatus 2 (62) are malfunctioning, it is possible to switch connections between the measuring units 6-1, 6-2, 64-1 and 64-2, and the water treatment line 112 by a switching mechanism to measure ballast water only by either the measuring units 6-1 and 64-1 or the measuring units 6-2 and 64-2, for example, so that ballast water in which neutralizing agent is insufficiently injected is prevented from being discharged.

The measurement apparatus 2 (62), the ballast water treatment equipment 102 and the connection between them are similar to those of the third embodiment, and description thereof will be omitted.

[Advantageous Effects of the Other Embodiments]

The control unit 14 can output an instruction to adjust the amount of oxidant or neutralizing agent to be added and directly control pumps or control valves. An instruction route for the pumps or the control valves is simplified, and a burden on the control unit 110 of the ballast water treatment equipment 102 can be reduced. If the control unit 14 outputs alarm information or a signal to stop the ballast water treatment equipment for excessive addition of the oxidant, the excessive addition of the oxidant is prevented, and it is possible to control the amount of oxidant used. If the control unit 14 outputs alarm information or a signal to stop the ballast water treatment equipment for insufficient addition of the neutralizing agent, it is prevented that insufficiently neutralized ballast water is drained into the sea, and it is possible to control the amount of oxidant discharged into the sea. If the control unit 14 directly outputs one or more of a result of water quality measurement of ballast water, and alarm information or information to stop the ballast water treatment equipment to the management apparatus on the ship side, or outputs them to the ship management center established on land through the communication line, it is possible to reduce a burden on the control unit 110 on the ballast water treatment equipment side.

[Modification]

The judgment about insufficient addition of the oxidant may be omitted. Insufficient addition of the oxidant can be adjusted by adding the oxidant into the ballast tank.

As described above, the most preferred embodiments of the present invention and the like have been described. The present invention is not limited to the above description. One skilled in the art can make various modifications and changes based on the spirit of the invention described in the claims or disclosed in the specification. It goes without saying that such modifications and changes are included in the scope of the present invention. For example, in a case where time until discharge after addition of the oxidant is short, control may be performed to omit measurement of the oxidant concentration before addition of the neutralizing agent, add the neutralizing agent, regarding the concentration of the added oxidant as the oxidant concentration before addition of the neutralizing agent, and perform only confirmation about whether neutralization is performed, by the second measuring unit.

### INDUSTRIAL APPLICABILITY

The present invention is capable of, in ballast water treatment for sterilizing organisms in ballast water by adding an oxidant such as sodium hypochlorite and ozone to the ballast water, measuring the water quality of the ballast water, for example, a TRO concentration. The present invention is useful for water quality measurement of ballast water in a ship where ballast water treatment is performed and is, additionally, useful for water quality measurement of other kinds of waters to which chemicals such as an oxidant and a neutralizing agent for the oxidant are added.

### DESCRIPTION OF REFERENCE NUMERALS

2, 62 Measurement apparatus
4 Casing
6-1, 6-2, 64-1, 64-2 Measuring unit
8 Draining unit
10 Switching unit
12 Display input unit
14 Control unit
20-1 High concentration measuring instrument
20-2 Low concentration measuring instrument
22-1, 22-2 Water supply pipe
24-1, 24-2 Drainage pipe
26-1, 26-2, 26-3, 26-4 Flow rate adjusting unit
28-1, 28-2, 28-3 On-off valve
30-1, 30-2, 30-3 Constant flow rate valve
32, 33 Connection pipe
34-1, 34-2, 34-3, 34-4, 34-5, 34-6 Switching valve
50 Dilution water supplying unit
52 Dilution water pipe
54 Branch pipe
56-1, 56-2 Pressure adjusting unit
58-1, 58-2 Backflow preventing unit
62 Measurement apparatus
64-1, 64-2 Measuring unit

1. A ballast water measurement apparatus comprising:
a first measuring unit for measuring an oxidant concentration of ballast water after addition of an oxidant or ballast water before addition of a neutralizing agent;
a second measuring unit for measuring an oxidant concentration of ballast water after neutralization of the oxidant; and
a casing that accommodates the first measuring unit and the second measuring unit; wherein
oxidant concentration measurement ranges of the first measuring unit and the second measuring unit are different.

2. The ballast water measurement apparatus according to claim 1, comprising a switching unit that is connected to the first measuring unit and the second measuring unit and that is for switching supply destinations of ballast water supplied to a first supply pipe of the first measuring unit or a second supply pipe of the second measuring unit.

3. The ballast water measurement apparatus according to claim 1, wherein both or one of the first measuring unit and the second measuring unit includes a flow rate adjusting unit for adjusting a flow rate of ballast water to be collected, and measures an oxidant concentration of the ballast water adjusted by the flow rate adjusting unit.

4. The ballast water measurement apparatus according to claim 1, comprising a dilution water supplying unit that is connected to the first measuring unit and that is for supplying dilution water to the first measuring unit.

5. The ballast water measurement apparatus according to claim 4, wherein the dilution water supplying unit comprises a dilution water pipe and a third flow rate adjusting unit for adjusting a flow rate of the dilution water flowing in the dilution water pipe.

6. The ballast water measurement apparatus according to claim 1, wherein
both or one of the first measuring unit and the second measuring unit includes a pressure adjusting unit, and the pressure adjusting unit adjusts a pressure of ballast water in a first water supply pipe of the first measuring unit or a second water supply pipe of the second measuring unit.

7. The ballast water measurement apparatus according to claim 1, comprising
a control unit for receiving both or one of a measurement result of the first measuring unit and a measurement result of the second measuring unit to generate an output signal of the both or one of the measurement results; wherein
the output signal includes information about the oxidant concentration, adjustment information about the amount of the oxidant added, adjustment information about the amount of the neutralizing agent added for neutralizing the oxidant, alarm information or a signal to stop ballast water treatment.

8. A ship comprising the ballast water measurement apparatus according to claim 1.

9. A ballast water measurement method comprising:
a process for measuring an oxidant concentration of ballast water after addition of an oxidant or ballast water before addition of a neutralizing agent, by a first measuring unit in a casing; and
a process for measuring an oxidant concentration of ballast water after neutralization of the oxidant, by a second measuring unit in the casing; wherein
oxidant concentration measurement ranges of the first measuring unit and the second measuring unit are different.
